SEQUENCE LISTING (52 sequences)

SEQ ID NO: 1 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 2 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 222 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 4 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 5 - protein, 536 aa, synthetic construct
MSSSFLSSTAFFLLLCLGFCHVSSQLRPRERPRGCPCTGRASSLARDSAAAASDPGTIVHNFSRTEPRTEPAGGSHSGSSSKLQALFAHPLYNVPEEPPLLGAEDSLLASQEALRYYRRKVARWNRRHKMYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGNPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQLAPDSGQANLTSHDEL

SEQ ID NO: 6 - protein, 586 aa, synthetic construct
MSSSFLSSTAFFLLLCLGFCHVSSLDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARHDEL

SEQ ID NO: 7 - DNA, 426 bp, Cauliflower mosaic virus
  source 1..426: mol_type = genomic DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 8 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 9 - DNA, 714 bp, Agrobacterium tumefaciens
  source 1..714: mol_type = genomic DNA
gtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 10 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 11 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 12 - protein, 210 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 13 - protein, 439 aa, Gallus gallus
MGALLALLDPVQPTRAPDCGGILTPLGLSYLAEVSKPHAEVVLRQDLMAQRASDLFLGSMEPSRNRITSVKVADLWLSVIPEAGLRLGIEVELRVAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVSVVVHKDYVLVPCKLKLRSTMA

SEQ ID NO: 14 - protein, 1463 aa, Bos taurus
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 15 - DNA, 1341 bp, Glycine max
  source 1..1341: mol_type = genomic DNA
ggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 16 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 17 - protein, 517 aa, Gallus gallus
MTDWVLHHKVGPLDMTTRYIFPLLPLPFLPHSESKRAVCAPRCSAMRTARQFVQVALALCCFADIAFGIEVNCSLYASGIGKDGTSWVACPRNLKPVCGTDGSTYSNECGICLYNREHGANVEKEYDGECRPKHVTIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 18 - protein, 225 aa, Gallus gallus
VVSTAAPTDPRRRMAVSVLLFISVALAGVLSTASQACKLEPVKIDLANPQLAGKWYFIQVATEVELYQARFANIDNSYFISTPDVKLNKTNIKEYSQLGDLCLSTNTDYVVLENGYEFTDGAKNINNFRIIKSKIDNMLIIDYQYQIEKMDYHMLTLFKRNPTASKEEMEIFESYTKCLGLDKDKIKAFPRNKSECKEEKQINSFNATTQAQDFLEEKVLQNRNI

SEQ ID NO: 19 - DNA, 886 bp, Glycine max
  source 1..886: mol_type = genomic DNA
gtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagatcacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatc

SEQ ID NO: 20 - protein, 208 aa, Gallus gallus
ALTSGLGPDVYQFLQDMGMKFFTNSKIRQPTVCTRETVRPPSYFLNAGFTASTHHVKLSAEVAREERGKRHILETIREFFPETWIWDIILINSTGKASVSYTIPDTITEWKASAFCVLPPNVVEESARASVSVLGDILGSAMQNTQNLLQMPYGCGEQNMVLFAPNIYVLDYLNETQQLSEDMKSKTIGYLESGYQKQLSYKHPDGSY

SEQ ID NO: 21 - protein, 152 aa, Gallus gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 22 - protein, 148 aa, Gallus gallus
MVGSPRAPLLLLASLIVALALALAVSPAAAQGPRKGRLLGGLMEADVNEEGVQEALSFAVSEFNKRSNDAYQSRVVRVVRARKQVVSGMNYFLDVELGRTTCTKSQANLDSCPFHNQPHLKREKLCSFQVYVVPWMNTINLVKFSCQD

SEQ ID NO: 23 - DNA, 62 bp, Tobacco mosaic virus
  source 1..62: mol_type = genomic DNA
atttttacaacaattaccaacaacaacaaacaacaaacaacattacaattacatttacaatt

SEQ ID NO: 24 - DNA, 739 bp, Arabidopsis thaliana
  source 1..739: mol_type = genomic DNA
ttgcttcttctctctttttttggttcaatatgaaccttttgatgtccactatccttttatataatgaaatgataaggtttttgatatgttatgtggttcttgataacattatacaattacttaatatctacatatgaaaggttggaattttttttaagtcaccacaatagaggtgacacgtgtaagcacctcgttaatcttatctcatccaagatgggggtaggaagaggatatgaatgtatgattgaggttggttttgagttttttttttttttttttttgagtccttcaacttgttattttaatttttttttggtgggggaggaggggggttgaaatatttatcatatagtagtccaaagtaaattgatagctagagtacttgtttgcttgcttatattgtcctcaactttatgtaataccatgattccaacttagacactcttttaagttgtaattttcattattttctttttttagagttttatgttgaattcgcataattttcaatcggataatacaagaaaaataatattttagtataattttatacatgaaatttcgggaaggtaggatatacggattgtttgtcggatcagagactttactcgtacctttgtaactgttgatcccaaatacagatagtgactccagagttatatattatacctatagagactaatatgatttagtgttattaaaattagtattactaattaattgtaatgccccatgaatt

SEQ ID NO: 25 - DNA, 4968 bp, synthetic construct
  misc_feature 1..4968: note = Description of Artificial Sequence: Synthetic polynucleotide
  source 1..4968: mol_type = other DNA
tctgtgaagacaaactagaattcgagctcggagtgagaccgcagctggcacgacaggtttgccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgacggtctcacgctttacttgtcttctgcacgaagtggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatcaggcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcaagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggatctgccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattggccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggagatcctgatctgactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccac

SEQ ID NO: 26 - DNA, 790 bp, synthetic construct
  source 1..790: mol_type = other DNA
gccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctg

SEQ ID NO: 27 - DNA, 618 bp, synthetic construct
  source 1..618: mol_type = other DNA
ccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 28 - DNA, 537 bp, synthetic construct
  source 1..537: mol_type = other DNA
ccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatcc

SEQ ID NO: 29 - DNA, 128 bp, synthetic construct
  source 1..128: mol_type = other DNA
aaactatcagtgtttgacaggatatattggcgggtaaacctaagagaaaagagcgtttattagaataatcggatatttaaaagggcgtgaaaaggtttatccgttcgtccatttgtatgtgcatgcca

SEQ ID NO: 30 - DNA, 15 bp, synthetic construct
  source 1..15: mol_type = other DNA
cacgacgaattgtaa

SEQ ID NO: 31 - DNA, 1674 bp, synthetic construct
  source 1..1674: mol_type = other DNA
ctagatttactcccccgactggagcgacgtggagcgcgtccgtctggcgaaccaggatgttcttgtgcgcaaccagctgcggaagtcgctgcgccaggttgggcacaagttcgagggaggcctggggagccaccagctgctagctccgctgcgggcgatgccggatggccaaataaacacactctaagaatactacaggacttctcaagcgacccttcctctaatctctcatcacactcacttgagaagttacctcccgcagccgagccggccgagcgagcgcttagggggcgtgaccccggggcattacgacctcacgatcctgcacacaggcctttgttacgtgatccagggccgcgaagaagcgaatcccctcctggacccggaggcgacgcatcccttctggcgaggttatttgaacacccattgtacagagtcgccgtgcctcccctgactgaagaggacgtcttgttcaacgttaactctgacaccagacttagtcctaaagcggccgagaatcctgattggccgcatgccggggcagagggcgctgagttccttagcccaggagaggccgcggtggactcctatccgaactggttgaaattccatattggaataaatcgatacgaactatattctcgacacaatccggccatcgaggccctattacatgatctgtcctcacaacgtattacgtcagtggccatgaagagcggcggcacgcaactaaagctgattatgacatttcaaaactatggtcaagccctgtttaagccaatgaagcagacaagagaacaggagacgcctcccgacttcttctactttagtgattatgaacgacataatgctgaaatagcggcctttcacctagacaggatcttagacttccgtagggttcccccagttgcaggtaggatggttaacatgactaaagagataagggatgtaaccagggataagaagctatggaggacgttctttatttctcctgcgaacaatatctgtttttacggcgagtgctcctactattgcagcacagaacatgcactatgtggaaagcctgaccagattgaaggttccctggcggcctttcttccagacctctctcttgcgaaacgtaaaacttggcgtaatccatggagacgaagctatcacaaacgtaagaaagcggagtgggaggtagaccccgattactgcgaggaggttaagcaaaccccgccgtatgactctagccaccgaatattagacgttatggatatgacgatctttgattttcttatgggcaacatggatcgtcaccactacgaaacgttcgagaagtttggaaatgagacgtttattatccatctggacaacggcagaggctttgggaaatatagtcatgacgaattatccattcttgttcctttacagcaatgctgcagaatcaggaaatcaacgtatttacgtctccaactcctggcaaaagaggaatataagcttagcctcctaatggctgagtctctgaggggcgaccaagttgcgcctgttctgtatcaaccacatttagaagcacttgatagacgtttgcgtgttgtacttaaagctgtccgagactgtgtcgagagaaatggccttcattcagttgtggacgacgatctagataccgaacatagggcagcgtccgcacgt

SEQ ID NO: 32 - DNA, 1524 bp, synthetic construct
  source 1..1524: mol_type = other DNA
caactcaggccaagggaacgtccccgtgggtgcccgtgtaccggaagggcatctagtttggcgcgagattctgctgctgcggcgtcagacccagggacaattgtgcataatttttcccgtactgagccaagaacagagccggccggtgggagccatagcggaagttcatctaagttgcaagcactattcgcgcatcctttgtataatgtacctgaagaacctcctcttttaggagcggaggactcactcttagccagccaggaagcccttagatactaccgtagaaaggttgcccgatggaaccgtaggcataaaatgtaccgtgagcaaatgaatttaactagtcttgatcctcctctccaactgaggcttgaggctagttgggttcagttccatttgggtatcaatcgacacggcctttactctaggtcaagtcctgtggtatctaaacttcttcaagacatgcgtcattttccgacaatatctgccgattactcccaggatgagaaggctttgcttggagcatgtgactgtacccagatagtaaaacctagtggcgtccacttaaaactggtcctccgttttagcgattttgggaaagctatgtttaaacccatgcgacaacaacgtgatgaagaaacgccagttgatttcttctacttcattgacttccagcgtcacaacgcggagatagcagcattccacttagacagaattctagactttcgtcgagtcccccctaccgttggccgtatagtgaatgtaactaaagagattttggaggtgacaaaaaacgagatcttacaaagcgtcttttttgtatccccggcctccaacgtctgtttttttgcgaaatgtccctatatgtgcaaaactgaatatgcggtctgcggcaacccccatttactcgaaggtagtctcagtgcatttctccccagtctcaacttagctccacgtctaagcgtgccaaacccttggattaggagctatacgctagcgggcaaggaggagtgggaggtgaacccactttattgtgacacagtcaagcaaatctatccttataataactcacagagactactcaatgtcattgatatggctatcttcgatttcctgataggaaacatggataggcaccattatgagatgttcaccaagttcggggacgatggttttctgatacatctagacaacgcgcgtggcttcgggcgacactcccacgacgaaatctccattcttagccccctgagccagtgttgcatgatcaaaaagaaaacacttctgcatcttcagctcctcgctcaagctgactatcgactttccgacgtgatgcgagaatcactgcttgaagatcagctcagcccagtgcttactgaaccgcacctactggcactagatcgtagattacagacgatcttgaggacagtcgaggggtgcatcgttgctcacggccaacaaagcgtcatagttgatgggcctgtggaacagctagctccggattccggacaagcaaatttaaccagc

SEQ ID NO: 33 - DNA, 1674 bp, synthetic construct
  source 1..1674: mol_type = other DNA
ctcctacccaaattagaacgatctgctgcacgtccgagcggcgaacctggctgtagctgcgcacagccagctgccgaagctgccgcgcctggatgggctcaagctaggggtcatcccggtggagaacttgaagcggccgctagcgccgccggggatgcaggctggccaaataagcacactctgaggattctgcaagacttcagttccgaccccagttccaacctaacgagccactcactggaaaagctgcctccggctgccgaagctgcggaaggtgcaccgccaggccaagatccaggagttcgtagacctcccgacccagcgcataggccactcccgcgagatccgggtcctagaggccctgtcttgcccccaggtcttagcggagacgggtccttacttacgcgtcttttccaacacccgctataccaggtgcccataccgcccctaacagaaggcgatgttctctttaatgtcaatagcgatataagattcaaccccaaagctgcaaccgccgagaacccagattggccacacgaggggccggaagatgagtttttacctactggtgaagcggcagttgactcttacccgaattggctgaagtttcatattgggatcaatagatacgagctttacagccgacataatccggccgtgggagcgctcttacaagacctcgggacgcaaaagattacttctgtcgctatgaaatctggcgggacacagctcaaacttattatgactttccagaattatggccaagctctgttcaagccgatgaagcagactagagagcaggagacaccccctgacttcttctacttcagcgactatgaaaggcataatgcagaaattgcggcattccaccttgataggatcttagacttccgaagggtaccaccggtagcaggtagactagtcaatatgactaaagagattagagatgtcactcgtgacaagaaactatggcgtacattctttataagccctgctaacaatgtatgcttttatggcgaatgttcttactattgctctacagaacatgcactgtgtggaaaacccgaccagattgaggggtcactagccgcatttctgccagacttggcattggccaagcgtaagacgtggcgtaatccgtggcgacgtagttaccacaagagaaagaaggcggagtgggaagtagacccagactactgcgaggaggttagacaaacacctccatatgattctagtcatagactgttggatgttatggacatgacaatttttgattttctcatggggaacatggatcgtcaccactacgaaacctttgagaaattcggcaatgagacattcattatccacttagataatggtcgaggttttggcaaacacagccatgacgaactatctatattagtgcctttacaacagtgctgtagaatccgaaggtctacctatttgagacttcaactgttggcccaagaggagcatcgtctatcacttttaatggccgaggctctaagggctgatcgtgtggctcccgtactctttcagcctcacttagaggctttagatcgtcgacttcgtatagtgcttcgagcggtaggcgattgcgtggagaaagatggactgcacagtgttgtagaggatgatttggggcctgagcacagggcggccgcgggacgt

SEQ ID NO: 34 - DNA, 1503 bp, synthetic construct
  source 1..1503: mol_type = other DNA
caccttggtcccagggtacgatccagactgcaaccgagggaacgaccgttggggtgcccttgtgcgcgtcgtgccgctagtcctgctccgggacctgccccgagtgcgcccaggcgtgtggaaccaagcggcggtggcgatccagggtccaaactcagggcacttttcgcacacccattgtataacgaaccagaggaacctccgctgcttgggcccgaggatagcctgctggcgggtcccgaagcattacgttattaccgaagaaaagtagcaaggtggaatagacgtcgaaagatgtacaaagaacaattaaacctaaccagcccagaaccgccggtgcaattgagacaagaggcatcatgggtacaatttcacttggggatcaataggcatgggctgtatcctcgtagctctccggtggtttctaaactccttcaggatatgagacacttccctacgatcagcgcggattatagccaggacgaaaaggctcttcttggcgcatgtgactgttctcaaattgttaagccctccggcgttcatctgaagctcgtacttcgtttttctgattttgggaaggcgatgtttaaacccatgagacagcagagggacgaggagacgccggaggacttcttctatttcatcgacttccagagacataatgccgaaattgctgccttccatctagatcgtattttggactttagacgtgtcccgccaacagtagggaggctcgtaaatgtgacaagggaaatactagaagtcactcgtaatgagattttacaaagcgtgtttttcgtgagtccggcaaacaatgtgtgcttctttgcgaaatgcccttatatgtgcaagacggagtatgctgtttgcgggtcaccccacctactagaaggttctttaagtgccttccttcccagcctcaacctcgctccacgtttcagtatgcctagtccatggatcaggtcatattccctagctggtcgtgaagagtgggaggtaaacccgctatactgcgaagcggtgaaacaggcttacccccacaatagcagttcacgtctattaaatattatcgatatggcgatcttcgactttctaatagggaacatggacaggcaccactacgaaatgttcactaagttcggtgaggacgggttcctaatacatttggataatgcccgtggttttgggagacattcacacgatgaagtctctattttagcccctttattccaatgttgtaggataaaaagaaaaaccctgctgcatcttcaactcctggctcaggctgattaccgtctttcagacgttatgcgtgagtctttgctagaggaccaattgagtcctgtactaacagaaccacatctgctagctttggacagacgtttgcagactgtgctaagaacggtccaggattgcatcgaggcccatggggaacaatcagtggtagccgacggcccggtgggccaatgggctcccgacagtagtagagcgaacgcaacttct

SEQ ID NO: 35 - DNA, 383 bp, Agrobacterium tumefaciens
  source 1..383: mol_type = genomic DNA
atttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcg

SEQ ID NO: 36 - DNA, 72 bp, synthetic construct
  source 1..72: mol_type = other DNA
atgagttctagctttttatcatccactgcgttcttcctgctcctttgcttaggcttctgccatgtgtcatct

SEQ ID NO: 37 - DNA, 63 bp, synthetic construct
  source 1..63: mol_type = other DNA
atggcaaacaagctcttcctcgtctgcgcaactttcgccctctgcttcctcctcaccaacgct

SEQ ID NO: 38 - DNA, 63 bp, synthetic construct
  source 1..63: mol_type = other DNA
tcgtacccctacgacgttcctgactacgcccatcatcaccatcaccaccatgatgagttgtag

SEQ ID NO: 39 - DNA, 714 bp, Agrobacterium tumefaciens; identical to SEQ ID NO: 9
  source 1..714: mol_type = genomic DNA

SEQ ID NO: 40 - DNA, 732 bp, synthetic construct
  source 1..732: mol_type = other DNA
atgggtggtggaggaagtggaggtggaggaagtgtcagtaaaggagaagctgtcataaaggagttcatgaggtttaaggtacacatggaagggtcaatgaacgggcatgaatttgagatcgagggggagggagaaggaaggccttatgagggcactcaaactgctaagttgaaggtcactaagggggggcctcttcctttttcttgggacatactcagccctcagttcatgtatggctcaagggcctttacaaaacaccctgccgatatccccgattactataagcagagctttcccgaaggcttcaagtgggagagggttatgaattttgaagatggcggtgctgttacagtcacacaggacacaagtcttgaggatgggaccctgatttataaagttaagctcagggggacaaatttcccaccagatggaccagtcatgcagaagaaaactatgggatgggaagcctctaccgagcgtctttatcccgaagatggcgttcttaaaggcgatattaagatggctttgaggctcaaagatggcggtcgttacctggccgatttcaaaaccacttacaaagccaaaaaaccagtacagatgccaggcgcttacaacgttgatcgtaagctggacatcacttcccacaacgaggattacacagttgttgaacagtatgagagatcagagggtagacatagtaccggcggaatggatgagctgtacaaaagttcg

SEQ ID NO: 41 - DNA, 263 bp, Agrobacterium tumefaciens
  source 1..263: mol_type = genomic DNA
gtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 42 - DNA, 627 bp, synthetic construct
  source 1..627: mol_type = other DNA
agagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtt

SEQ ID NO: 43 - DNA, 507 bp, synthetic construct
  source 1..507: mol_type = other DNA
caggagcaaaaccaagagcaacctattcgttgtgaaaaagacgagagattttttagtgataaaattgcaaagtacatcccaatccagtacgttttaagtcgatatccgagctacggtcttaattactatcaacagaagccagttgccttgataaacaaccagtttctcccatatccctactatgctaagccggccgccgtccgttctcctgctcagatattgcagtggcaggttttatccaacactgtcccggcgaagtcttgccaggcgcaacccaccaccatggctagacacccgcatccgcacctttcctttatggctattcctcccaaaaagaaccaagacaagacagagatcccgaccataaatactatcgcttctggtgagccaacttcaacaccgactattgaagcggtggagagcacggtcgccacacttgaagcatccccagaggtgactgaatcacccccggagataaacacggtacaggtcacctcaacggctgtt

SEQ ID NO: 44 - DNA, 507 bp, synthetic construct; identical to SEQ ID NO: 43
  source 1..507: mol_type = other DNA

SEQ ID NO: 45 - protein, 241 aa, synthetic construct
MANKLFLVCATFALCFLLTNARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWSSYPYDVPDYAHHHHHHHDEL

SEQ ID NO: 46 - protein, 250 aa, synthetic construct
MANKLFLVCATFALCFLLTNARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVSYPYDVPDYAHHHHHHHDEL

SEQ ID NO: 47 - protein, 210 aa, synthetic construct
MANKLFLVCATFALCFLLTNAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVTESPPEINTVQVTSTAVSYPYDVPDYAHHHHHHHDEL

SEQ ID NO: 48 - DNA, 60 bp, synthetic construct
  source 1..60: mol_type = other DNA
atggccaagctagttttttccctttgttttctgcttttcagtggctgctgcttcgcagca

SEQ ID NO: 49 - DNA, 24 bp, synthetic construct
  source 1..24: mol_type = other DNA
gattacaaagatgacgacgataag

SEQ ID NO: 50 - protein, 236 aa, synthetic construct
MAKLVFSLCFLLFSGCCFARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWDYKDDDDKHHHHHHHDEL

SEQ ID NO: 51 - protein, 246 aa, synthetic construct
MAKLVFSLCFLLFSGCCFARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVDYKDDDDKHHHHHHHDEL

SEQ ID NO: 52 - protein, 207 aa, synthetic construct
MAKLVFSLCFLLFSGCCFAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVTESPPEINTVQVTSTAVSDYKDDDDKHHHHHHHDEL

(19) United States
(12) Patent Application Publication
(10) Pub. No.: US 2025/0034219 A1
(43) Pub. Date: Jan. 30, 2025

(54) COMPOSITIONS, METHODS AND SYSTEMS FOR PHOSPHORYLATION OF PROTEINS IN PLANTS

(71) Applicant: Mozza Foods, Inc., Los Angeles, CA (US)

(72) Inventors: CORY J TOBIN, Los Angeles, CA (US); Noah Brookes, Los Angeles, CA (US)

(73) Assignee: Mozza Foods, Inc., Los Angeles, CA (US)

(21) Appl. No.: 18/709,465

(22) PCT Filed: Nov. 17, 2022
(86) PCT No.: PCT/US2022/080041

§ 371 Date: May 10, 2024

Related U.S. Application Data
(60) Provisional application No. 63/281,069, filed on Nov. 18, 2021.

(51) Int. Cl. C07K 14/47; A23C 11/08; A23C 20/00; A23J 1/00; A23J 1/14; A23J 3/14; A23J 3/16; C12N 9/12; C12N 15/82

(52) U.S. Cl. CPC C07K 14/4732; A23C 11/08; A23C 20/005; A23J 1/006; A23J 1/14; A23J 3/14; A23J 3/16; C12N 9/12; C12N 15/8257; C12Y 207/11001

(57) ABSTRACT

Compositions, methods and systems for phosphorylation of proteins in plants. One or more vectors are used to express at least one kinase and at least one payload protein in a plant cell, wherein the at least one payload protein is phosphorylated by the at least one kinase in the plant cell. For example, one or more vectors are used to co-express a recombinant kinase, a recombinant β-casein, and at least one of a recombinant κ-casein, recombinant αS1-casein, or a recombinant αS2-casein, wherein at least one of the recombinant caseins (e.g., the recombinant β-casein) is phosphorylated by the recombinant kinase in the plant cell.

### CROSS-REFERENCE

This application claims the benefit of International Patent Application Serial No. PCT/US2022/080041, filed on Nov. 17, 2022, which claims the benefit of U.S. Provisional Application Ser. No. 63/281,069, filed on Nov. 18, 2021, which application are each incorporated herein by reference in its entirety.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### INCORPORATION BY REFERENCE OF SEQUENCE LISTING

The present application is being filed along with a Sequence Listing in electronic format. The Sequence Listing is provided as a file entitled MOZZA-704-1-US_SEQ_LIST.xml, created on May 7, 2024, which is 74.3 kilobytes in size. The information in the electronic format of the Sequence Listing is incorporated by reference in its entirety.

### BACKGROUND

Protein phosphorylation is a post-translational modification of proteins in which a phosphate group is added to an amino acid in the protein. Chemical phosphorylation of food proteins can be achieved by using chemicals. However, chemical phosphorylation disrupts the native structure of food proteins because of the harsh reaction conditions. Moreover, unwanted chemical reagents from the final product can be difficult to remove. Enzymatic phosphorylation with ATP is a more desirable method to phosphorylate food proteins due to improved food safety. However, this method does not fit the needs of industrial-scale production due to the high cost of ATP and enzymes.

### SUMMARY

The current disclosure provides compositions, methods and systems for phosphorylation of proteins in plants. Described herein, in some aspects, are compositions comprising a deoxyribonucleic acid (DNA) encoding at least a portion of a recombinant kinase protein; and a recombinant κ-casein and at least one of a recombinant αS1-casein, a recombinant αS2-casein, or a recombinant β-casein. In some cases, the deoxyribonucleic acid (DNA) sequence encoding at least the portion of the recombinant kinase protein is at least 50%, at least 60%, at least 70%, at least 80%, at least 90%, at least 95%, or at least 99% of the deoxyribonucleic acid (DNA) sequence encoding the recombinant kinase protein. In some cases, the portion of a recombinant kinase protein is at least 50%, at least 60%, at least 70%, at least 80%, at least 90%, at least 95%, or at least 99% of the recombinant kinase protein. In some cases, the at least one of the recombinant caseins is phosphorylated. For example, the κ-casein is phosphorylated, the β-casein is phosphorylated, the αS1-casein is phosphorylated, or the αS2-casein is phosphorylated, or any combination thereof. In some cases, the recombinant β-casein is phosphorylated at S50. In some cases, the at least one of the recombinant caseins is phosphorylated by a kinase protein in a plant cell. In some cases, the recombinant κ-casein and at least one of a recombinant αS1-casein, a recombinant αS2-casein, or a recombinant β-casein form a casein micelle.

In some instances, the disclosed compositions further comprise one or more soy proteins. In some cases, the one or more soy proteins comprise at least 50%, at least 60%, at least 70%, at least 80%, at least 90%, or at least 95% total protein content (by weight) in the composition. In some cases, the soy protein is a storage protein comprising at least one of β-conglycinin (7s) and glycinin (11s). In some cases, the one or more soy proteins comprise at most 40%, at most 30%, at most 20%, at most 10%, at most 5%, or at most 1% total protein content (by weight) in the composition. In some cases, the one or more soy proteins comprise at most 0.5%, at most 0.4%, at most 0.3%, at most 0.2%, or at most 0.1% of total protein content (by weight) in the composition. In some cases, the recombinant kinase protein comprises at least 50%, at least 60%, at least 70%, at least 80%, at least 90%, at least 95%, or at least 99% sequence identity to Casein Kinase II, a tyrosine kinase, Fam20C or Fam20A. In some cases, the recombinant casein proteins comprise less than 40%, less than 30%, less than 20%, less than 15%, less than 10%, less than 5%, or less than 1% of total protein content (by weight) in the composition. In some cases, the recombinant κ-casein is at least 10%, at least 20%, at least 30%, at least 40%, or at least 45% of total casein abundance (by weight). In some cases, the recombinant κ-casein is at most 40%, at most 30%, at most 20%, or at most 10% of total casein abundance by weight. In some cases, the recombinant κ-casein is about 46% of total casein abundance (by weight). In some cases, the recombinant αS1-casein is less than 60%, or less than 50%, or less than 40% of total casein abundance (by weight). In some cases, the recombinant αS1-casein is about 33% of total casein abundance (by weight). In some cases, the recombinant β-casein is phosphorylated at S50. In some cases, the composition is essentially free of αS2-casein. In some cases, the phosphorylation of recombinant αS1-casein protein is undetectable using mass spectrometry. In some cases, the disclosed compositions comprises a green fluorescent protein (GFP). In some cases, the green fluorescent protein (GFP) is less than 1% of total protein content (by weight) in the composition. In some cases, the deoxyribonucleic acid (DNA) encoding the recombinant kinase protein is present in the composition in trace amounts. In some instances, the deoxyribonucleic acid (DNA) encoding the recombinant kinase protein is detectable using a polymerase chain reaction (PCR) test.

Described herein, in some aspects, are dairy products or dairy product substitutes, comprising any of the compositions disclosed herein. In some cases, the dairy product or dairy product substitute resembles raw milk in terms of at least one of physical appearance (e.g., color), sensory perceptions (e.g., mouthfeel, fattiness, creaminess, homogenization, richness, smoothness, or thickness), viscosity, foaming behavior, agglutination capacity, or any combination thereof. In some cases, the dairy product or dairy product substitute is essentially free of milk proteins other than the recombinant casein proteins described herein.

Described herein, in some aspects, are nucleic acid molecules encoding a recombinant kinase protein, a recombinant κ-casein and at least one of a recombinant αS1-casein, a recombinant αS2-casein, or a recombinant β-casein. In some cases, the nucleic acid sequence is codon optimized for expression in a plant. In some cases, the plant is soybean. In some cases, the plant is a dicot plant selected from the group consisting of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus. In some cases, the plant is a monocot plant selected from the group consisting of turf grass, corn, rice, oat, wheat, barley, sorghum, orchid, iris, lily, onion, palm, and duckweed.

In some cases, the nucleic acid molecule is stably integrated into the genome of a plant cell. Described herein, in some aspects, are expression vectors comprising the nucleic acid molecules disclosed herein. Described herein, in some aspects, are plant tissues comprising the nucleic acid molecules disclosed herein. In some cases, the plant tissue is at least one of leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, or leaf sheath of the plant.

Described herein, in some aspects, is a plant cell co-expressing a kinase, a κ-casein, and at least one of a αS1-casein, a αS2-casein, or a β-casein; wherein the κ-casein or at least one of a αS1-casein, a αS2-casein, or a β-casein is phosphorylated by the kinase in the plant cell, and wherein the κ-casein and at least one of a αS1-casein, a αS2-casein, or a β-casein form a casein micelle in the plant cell.

Described herein, in some aspects, is a method of phosphorylating a casein protein, comprising using one or more vectors to co-express a kinase, a κ-casein, and at least one of a αS1-casein, a αS2-casein, or a β-casein, in a plant cell; wherein at least one of the caseins is phosphorylated by the kinase in the plant cell; and isolating at least one of the phosphorylated caseins from the plant cell. In some cases, the kinase, the κ-casein, and at least one of the αS1-casein, the αS2-casein, or the β-casein are expressed in the plant cell using the same vector. In some cases, the kinase, the κ-casein, and at least one of the αS1-casein, the αS2-casein, or the β-casein are expressed in the plant cell using different vectors. In some cases, the disclosed method further comprises purifying or enriching the at least one of the phosphorylated caseins isolated from the plant cell. In some cases, the phosphorylated casein enriched is β-casein. In some cases, the disclosed method further comprises removing a native plant protein from the isolated proteins. In some cases, the native plant protein is a soy protein.

Described herein, in some aspects, are compositions comprising a recombinant kinase protein, a recombinant β-casein, a recombinant κ-casein, and at least one of a recombinant αS1-casein or a recombinant αS2-casein, wherein the recombinant β-casein is phosphorylated by the kinase protein, and wherein the phosphorylated recombinant β-casein, the recombinant κ-casein, and at least one of a recombinant αS1-casein or a recombinant αS2-casein form a casein micelle. In some cases, the recombinant kinase protein comprises at least 80% sequence identity to Fam20C or Fam20A. In some cases, the recombinant casein proteins comprise less than 40%, less than 30%, less than 20%, less than 15%, less than 10%, less than 5%, or less than 1% of total protein content (by weight) in the composition. In some cases, the recombinant κ-casein is at least 10%, at least 20%, at least 30%, at least 40%, or at least 45% of total casein abundance (by weight). In some cases, the recombinant κ-casein is at most 40%, at most 30%, at most 20%, or at most 10% of total casein abundance by weight. In some cases, the recombinant κ-casein is about 46% of total casein abundance (by weight). In some cases, the recombinant αS1-casein is less than 60%, or less than 50%, or less than 40% of total casein abundance (by weight). In some cases, the recombinant αS1-casein is about 33% of total casein abundance (by weight). In some cases, the recombinant β-casein is phosphorylated at S50. In some cases, the composition is essentially free of αS2-casein. In some cases, the phosphorylation of recombinant αS1-casein protein is undetectable using mass spectrometry. In some cases, the disclosed compositions comprises a green fluorescent protein (GFP). In some cases, the green fluorescent protein (GFP) is less than 1% of total protein content (by weight) in the composition. In some instances, the disclosed compositions further comprise one or more soy proteins. In some cases, the one or more soy proteins comprise at least 50%, at least 60%, at least 70%, at least 80%, at least 90%, or at least 95% total protein content (by weight) in the composition. In some cases, the soy protein is a storage protein comprising at least one of β-conglycinin (7s) and glycinin (11s). In some cases, the one or more soy proteins comprise at most 40%, at most 30%, at most 20%, at most 10%, at most 5%, or at most 1% total protein content (by weight) in the composition. In some cases, the one or more soy proteins comprise at most 0.5%, at most 0.4%, at most 0.3%, at most 0.2%, or at most 0.1% of total protein content (by weight) in the composition.

Described herein, in some aspects, is a plant cell co-expressing a kinase, a recombinant β-casein, a recombinant κ-casein, and at least one of a recombinant αS1-casein or a recombinant αS2-casein, wherein the recombinant β-casein is phosphorylated by the kinase protein in vivo, and wherein the phosphorylated recombinant β-casein, the recombinant κ-casein, and at least one of a recombinant αS1-casein or a recombinant αS2-casein form a casein micelle in vivo.

Described herein, in some aspects, are methods of producing a composition, comprising using one or more vectors to co-express a kinase, a recombinant β-casein, a recombinant κ-casein, and at least one of a recombinant αS1-casein or a recombinant αS2-casein, in a plant cell; wherein the recombinant β-casein is phosphorylated by the kinase protein in vivo; and wherein the phosphorylated recombinant β-casein, the recombinant κ-casein, and at least one of a recombinant αS1-casein or a recombinant αS2-casein form a casein micelle in vivo; and isolating the phosphorylated recombinant β-casein, the recombinant κ-casein, and at least one of the recombinant αS1-casein or the recombinant αS2-casein from the plant cell. In some cases, the method further comprises purifying or enriching the proteins isolated from the plant cell. In some cases, the method further comprises removing a native plant protein (e.g., a soy protein) from the isolated proteins. In some cases, the method further comprises purifying or enriching one or more phosphorylated proteins (e.g., β-casein) in the isolated proteins. It is contemplated that the recombinant-casein, the recombinant κ-casein, and the at least one of the recombinant αS1-casein or the recombinant αS2-casein can be expressed in the plant cell using the same vector (i.e., all recombinant proteins are encoded by the same vector), or using different vectors (for example, one vector encoding the kinase, another vector encoding the case proteins).

Described herein, in some aspects, are nucleic acid molecules (e.g., vectors) for expressing a phosphorylated payload protein in a plant, wherein the nucleic acid molecules (e.g., vectors) may comprise at least one of a polynucleotide sequence encoding: a first kinase, a second kinase, a first payload protein, a promoter sequence, a terminator sequence, a second payload protein, and combinations thereof. In some instances, described herein are nucleic acid molecules (e.g., vectors) for expressing a phosphorylated payload protein in a plant, wherein the nucleic acid molecules may comprise, for example, a polynucleotide sequence encoding: a first kinase, a second kinase, a first payload protein, a promoter sequence, a terminator sequence, and optionally a second payload protein and/or a third payload protein.

Contemplated promoters include CaMV 35S, AtuMas Pro+5′UTR, RbcS2 promoter, a soybean GY1 Promoter, soybean CG1 Promoter, or other suitable promoters. Contemplated terminator sequences can be octopine synthase terminator (Ocst), Octopine (OCS) terminator, NOS terminator or other suitable terminator sequences. It is contemplated that the first or the second kinase can be a human kinase or a non-human kinase, for example, a bovine kinase. In some instances, at least one of the first and the second kinase is FAM20A, FAM20C, casein Kinase II or a tyrosine kinase. In some instances, at least one of the first kinase and the second kinase has at least 50%, 60%, 70%, 80%, 85%, 90%, 95%, or 99% sequence identity to SEQ ID: 5, SEQ ID: 6, or SEQ ID: 8. In some instances, the first kinase is different from the second kinase. For example, the first kinase can any one of the kinases mentioned herein, and the second kinase can be a different kinase mentioned herein.

In some instances, the first or second payload protein is a mammalian protein, for example, a human protein, a ruminant protein, a primate protein. In some instances, the ruminant animal includes, for example, a cow, a buffalo, a yak, a deer, a bovine, a goat, and a sheep.

In some instances, the first or second payload protein comprises a whey protein, including, for example, α-lactalbumin, β-lactoglobulin, serum albumin, immunoglobulins, and proteose peptone. In some instances, the payload protein comprises an egg white protein, including, for example, ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, avidin, and cystatin. In some instances, the egg white protein has at least 50%, 60%, 70%, 80%, 85%, 90%, 95%, or 99% sequence identity to the amino acid SEQ ID: 10, SEQ ID: 11, SEQ ID: 12, SEQ ID: 13, SEQ ID: 16, SEQ ID: 17, SEQ ID: 18, SEQ ID: 20, SEQ ID: 21, or SEQ ID: 22.

In some instances, the payload protein is a collagen protein, including, for example, Collagen I, Collagen II, Collagen III, Collagen IV, Collagen V, Collagen VI, Collagen VII, Collagen VIII, Collagen IX, Collagen X, Collagen XI, Collagen XII, Collagen XIII, Collagen XIV, Collagen XV, Collagen XVI, Collagen XVII, Collagen XVIII, Collagen XIX, Collagen XX, Collagen XXI, Collagen XXII, Collagen XXIII, Collagen XXIV, Collagen XXV, Collagen XXVI, Collagen XXVII, and Collagen XXVIII. In some instances, the collagen protein comprises one or more α chains, for example, wild type Bovine Collagen Alpha-1 (I) Chain. In some instances, the collagen protein expressed has at least 50%, 60%, 70%, 80%, 85%, 90%, 95%, or 99% sequence identity to the amino acid SEQ ID: 14.

In some instances, the first or second payload protein is a casein protein, including, for example, αS1-casein, αS2-casein, β-casein, and κ-casein. The casein protein can be from any mammalian species (including human) including from a ruminant animal. In some instances, the casein protein has at least 50%, 60%, 70%, 80%, 85%, 90%, 95%, or 99% sequence identity to the amino acid SEQ ID NO:1, SEQ ID NO:2, SEQ ID NO:3, SEQ ID NO:4, SEQ ID NO:42, SEQ ID NO: 43, SEQ ID NO:44, SEQ ID NO:45, SEQ ID NO:46, or SEQ ID NO:47. In some instances, the second payload protein is different from the first payload protein. For example, the first payload protein is κ-casein, and the second payload protein is at least one of αS1-casein, αS2-casein, and β-casein. It is contemplated that the same vector can express casein proteins from different species, for example, the first pay load protein is human κ-casein, and second pay load protein is a bovine αS1-casein, αS2-casein, or β-casein. As another example, the first pay load protein κ-casein is a bovine casein, and second pay load protein is a human β-casein.

In some aspects, the current disclosure also provides methods for expressing a

phosphorylated payload protein in a plant, comprising transforming the plant with a vector as described herein, and growing the transformed plant, wherein the payload protein is phosphorylated by the first or second kinase. In some instances, phosphorylation using the methods described herein leads to a higher yield or improved quality of food protein production in plants, compared to using an alternative method that does not use vectors described herein.

In some aspects, the current disclosure also provides methods of expressing a phosphorylated payload protein in a plant, comprising transforming the plant with a first vector, a second vector, and a third vector; and growing the transformed plant, wherein the payload protein is phosphorylated by the kinase; wherein the first vector comprises a first polynucleotide sequence encoding a first kinase, the second vector comprises a second polynucleotide sequence encoding a second kinase, and the third vector comprises a third polynucleotide sequence encoding the payload protein.

In some aspects, the current disclosure also provides food products and food product substitutes comprising the phosphorylated payload protein made using the method describe above. Contemplated food products include dairy products or products that resembles a dairy product (i.e., dairy product substitutes). The term “dairy product” as used herein refers to milk (e.g., whole milk (at least 3.25% milk fat), partly skimmed milk (from 1% to 2% milk fat), skim milk (less than 0.2% milk fat), cooking milk, condensed milk, flavored milk, goat milk, sheep milk, dried milk, evaporated milk, milk foam), and products derived from milk, including but not limited to yogurt (e.g., whole milk yogurt (at least 6 grams of fat per 170 g), low-fat yogurt (between 2 and 5 grams of fat per 170 g), nonfat yogurt (0.5 grams or less of fat per 170 g), greek yogurt (strained yogurt with whey removed), whipped yogurt, goat milk yogurt, Labneh (labne), sheep milk yogurt, yogurt drinks (e.g., whole milk Kefir, low-fat milk Kefir), Lassi), cheese (e.g., whey cheese such as ricotta; pasta filata cheese such as mozzarella; semi-soft cheese such as Havarti and Muenster; medium-hard cheese such as Swiss and Jarlsberg; hard cheese such as Cheddar and Parmesan; washed curd cheese such as Colby and Monterey Jack; soft ripened cheese such as Bric and Camembert; fresh cheese such as cottage cheese, feta cheese, cream cheese, and curd; processed cheese; processed cheese food; processed cheese product; processed cheese spread; enzyme-modulated cheese; cold-pack cheese), dairy-based sauces (e.g., fresh, frozen, refrigerated, or shelf stable), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (i.e., protein content of at least 80% by weight), milk protein isolate (i.e., protein content of at least 90% by weight), whey protein concentrate, whey protein isolate, demineralized whey protein concentrate, demineralized whey protein concentrate, .beta.-lactoglobulin concentrate, .beta.-lactoglobulin isolate, .alpha.-lactalbumin concentrate, .alpha.-lactalbumin isolate, glycomacropeptide concentrate, glycomacropeptide isolate, casein concentrate, casein isolate, nutritional supplements, texturizing blends, flavoring blends, coloring blends), ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, and bars. As used herein, the term “food product substitute” (e.g., “dairy product substitute”) refers to a food product that resembles a conventional food product (e.g., can be used in place of the conventional food product). Such resemblance can be due to any physical, chemical, or functional attribute. In some embodiments, the resemblance of the food product provided herein to a conventional food product is due to a physical attribute. Non-limiting examples of physical attributes include color, shape, mechanical characteristics (e.g., hardness, G′ storage modulus value, shape retention, cohesion, texture (i.e., mechanical characteristics that are correlated with sensory perceptions (e.g., mouthfeel, fattiness, creaminess, homogenization, richness, smoothness, thickness), viscosity, and crystallinity. In some embodiments, the resemblance of the food product provided herein and a conventional food product is due to a chemical/biological attribute. Non-limiting examples of chemical attributes include nutrient content (e.g., type and/or amount of amino acids (e.g., PDCAAS score), type and/or amount of lipids, type and/or amount of carbohydrates, type and/or amount of minerals, type and/or amount of vitamins), pH, digestibility, shelf-life, hunger and/or satiety regulation, taste, and aroma. In some embodiments, the resemblance of the food product provided herein to a conventional food product is due to a functional attribute. Non-limiting examples of functional attributes include gelling/agglutination behavior (e.g., gelling capacity (i.e., time required to form a gel (i.e., a protein network with spaces filled with solvent linked by hydrogen bonds to the protein molecules) of maximal strength in response to a physical and/or chemical condition (e.g., agitation, temperature, pH, ionic strength, protein concentration, sugar concentration, ionic strength)), agglutination capacity (i.e., capacity to form a precipitate (i.e., a tight protein network based on strong interactions between protein molecules and exclusion of solvent) in response to a physical and/or chemical condition), gel strength (i.e., strength of gel formed, measured in force/unit area (e.g., pascal (Pa))), water holding capacity upon gelling, syneresis upon gelling (i.e., water weeping over time)), foaming behavior (e.g., foaming capacity (i.e., amount of air held in response to a physical and/or chemical condition), foam stability (i.e., half-life of foam formed in response to a physical and/or chemical condition), foam seep), thickening capacity, use versatility (i.e., ability to use the food product in a variety of manners and/or to derive a diversity of other compositions from the food product; e.g., ability to produce food products that resemble milk derivative products such as yoghurt, cheese, cream, and butter), and ability to form protein dimers.

In some aspects, the current disclosure also provides plants transformed with a vector as described herein, wherein the payload protein is phosphorylated by the first or the second kinase in vivo in the plant. Contemplated plants can be a dicot plant, for example, Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus. Contemplated plants can also be a monocot plant, for example, turf grass, corn, rice, oat, wheat, barley, sorghum, orchid, iris, lily, onion, palm, and duckweed.

Described herein, in some aspects, are nucleic acid molecules (e.g., vectors) for expressing a phosphorylated casein protein in a plant. For example, the nucleic acid molecules (e.g., vectors) can comprise polynucleotide sequences encoding a kinase, κ-casein, and at least one of αS1-casein, αS2-casein, and β-casein. In some instances, the casein protein has at least 50%, 60%, 70%, 80%, 85%, 90%, 95%, or 99% sequence identity to the amino acid SEQ ID NO:1, SEQ ID NO:2, SEQ ID NO:3, SEQ ID NO:4, SEQ ID NO:42, SEQ ID NO:43, SEQ ID NO:44, SEQ ID NO:45, SEQ ID NO:46, SEQ ID NO:47, SEQ ID NO: 50, SEQ ID NO: 51, or SEQ ID NO: 52.

In some aspects, the current disclosure also provides methods of enhancing casein micelle formation in a plant, comprising transforming the plant with a vector as described herein and growing the transformed plant, wherein at least one of κ-casein, αS1-casein, αS2-casein, and β-casein is phosphorylated by the kinase.

In some aspects, the current disclosure also provides methods of enhancing casein micelle formation in a plant, comprising transforming the plant with a first vector and a second vector, and growing the transformed plant; wherein the first vector comprises a first polynucleotide sequence encoding a kinase, wherein the second vector comprises a second polynucleotide sequence encoding a κ-casein and at least one of αS1-casein, αS2-casein, and β-casein; wherein at least one of κ-casein, αS1-casein, αS2-casein, and β-casein is phosphorylated by the kinase, and wherein the κ-casein and at least one of αS1-casein, αS2-casein, and β-casein form the casein micelle in the plant in vivo.

In some aspects, the current disclosure also provides methods of enhancing casein micelle formation in a plant, comprising transforming the plant with a first vector, a second vector, and a third vector, and growing the transformed plant; wherein the first vector comprises a first polynucleotide sequence encoding a kinase, wherein the second vector comprises a second polynucleotide sequence encoding a κ-casein, wherein the third vector comprises a third polynucleotide sequence encoding at least one of αS1-casein, αS2-casein, and β-casein; wherein at least one of κ-casein, αS1-casein, αS2-casein, and β-casein is phosphorylated by the kinase, and wherein the κ-casein and at least one of αS1-casein, αS2-casein, and β-casein form the casein micelle in the plant in vivo.

In some aspects, phosphorylation using the methods described herein leads to improved micelle formation in plant cells, for example, in terms of increased number of micelles, micelles becoming more stable, and increased solubility of casein proteins. As a result, food products containing phosphorylated caseins made using the methods described herein have superior quality, including, for example, increased viscosity, melting point, and binding to calcium (e.g., calcium phosphate) than food products without phosphorylated caseins.

In some aspects, phosphorylation of a casein protein in a plant by using the nucleic acid molecules (e.g., vectors) and methods described herein increases the expression level of the casein protein in the plant, wherein the casein protein is selected form the group consisting of κ-casein, αS1-casein, αS2-casein, and β-casein, and wherein phosphorylation of a casein protein increases expression level of the casein protein by at least 10%, 20%, 30%, 40%, 50%, 60%, 70%, 80%, 90%, or even 100%.

In some aspects, phosphorylation of a casein protein in a plant by using the nucleic acid molecules (e.g., vectors) and methods described herein increases its ability to aggregate or bind to another casein protein, wherein the casein protein is selected form the group consisting of κ-casein, αS1-casein, αS2-casein, and β-casein. In some aspects, phosphorylation of a casein protein in a plant by using the nucleic acid molecules (e.g., vectors) and methods described herein improves casein micelle formation, by increasing the number of micelles, or by stabilizing the micelles, or both. In some aspects, phosphorylation of a casein protein in a plant by using the nucleic acid molecules (e.g., vectors) and methods described herein increases its binding to calcium by at least 10%, 20%, 30%, 40%, 50%, 60%, 70%, 80%, 90%, or even 100%.

In some aspects, phosphorylation of a casein protein in a plant by using the nucleic acid molecules (e.g., vectors) and methods described herein increases the viscosity of a liquid containing the phosphorylated casein proteins, compared to a solution containing same amount of unphosphorylated casein proteins.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 shows an example of an expression plasmid for the production of kinase proteins in plants.

FIG. 2 shows a map of a pMOZ12 expression plasmid, which is an example expression plasmid for the production of recombinant H. sapiens Fam20A in plants.

FIG. 3 shows a map of a pMOZ702 expression plasmid, which is an example expression plasmid for the production of recombinant B. taurus Beta Casein in plants.

FIG. 4 shows a map of a pMOZ14 expression plasmid, which is an example expression plasmid for the production of recombinant B. taurus Fam20C in plants.

FIG. 5 shows a map of a pMOZ15 expression plasmid, which is an example expression plasmid for the production of recombinant B. taurus Fam20A in plants.

FIG. 6 shows a map of a pMOZ700 expression plasmid, which is an example expression plasmid for the production of recombinant B. taurus Kappa Casein in plants.

FIG. 7 shows a map of a pMOZ701 expression plasmid, which is an example expression plasmid for the production of recombinant B. taurus αS1 Casein in plants.

FIG. 8 shows a map of a pMOZ1066 expression plasmid, which is an example expression plasmid for the production of recombinant B. taurus αS1, Beta, and Kappa Casein, and Fam20C in plants.

FIG. 9 shows a map of a vector backbone including a nucleic acid sequence encoding a β-lactamasc.

FIG. 10 shows results of Western blot showing the expression of recombinant Beta casein, HsFam20C, and HsFam20A in a systemically-infected N. benthamiana plant using combinations of the pMOZ11 (expresses HsFam20C), pMOZ12 (expresses HsFam20A) and pMOZ702 (expresses beta casein) expression plasmids.

FIG. 11 shows results of Western blot showing the expression of recombinant Beta casein, HsFam20C, and HsFam20A in a systemically-infected N. benthamiana plant using combinations of the pMOZ11 (expresses HsFam20C), pMOZ12 (expresses HsFam20A), pMOZ14 (expresses BtFam20C), pMOZ15 (expresses BtFam20A) and pMOZ702 (expresses beta casein) expression plasmids.

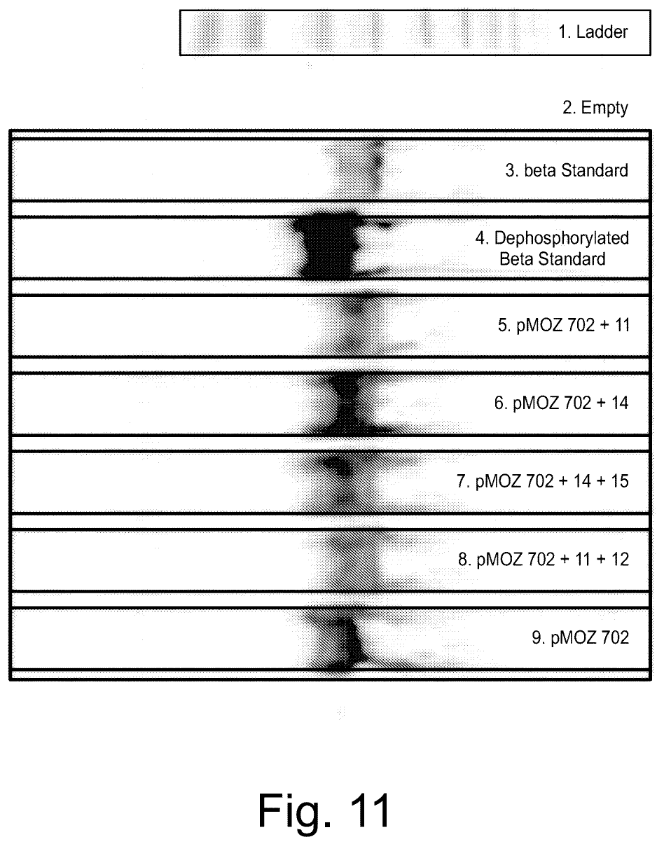

FIG. 12 shows the unique protein abundance of milk made from pMOZ1066 transiently transformed soybeans, with relative abundances of 1066, bovine, and soy milk.

FIG. 13 shows the normalized casein abundance of αS1, αS2, Beta, and Kappa Casein in 1066 milk and raw milk.

FIG. 14 shows the proportion of each casein out of the total casein abundance in 1066 soy milk and raw milk.

FIG. 15 shows the formation of casein micelles in vivo with recombinant kinase in soy protein storage vacuoles.

### DETAILED DESCRIPTION

The following embodiments are described in sufficient detail to enable those skilled in the art to make and use the invention. It is to be understood that other embodiments would be evident based on the present disclosure, and that system, process, or mechanical changes can be made without departing from the scope of an embodiment of the present disclosure.

In the following description, numerous specific details are given to provide a thorough understanding of the invention. However, it will be apparent that the invention can be practiced without these specific details. In order to avoid obscuring an embodiment of the present disclosure, some well-known techniques, system configurations, and process steps are not disclosed in detail. Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Definitions

A specified nucleic acid is “derived from” a given nucleic acid when it is constructed using the given nucleic acid's sequence, or when the specified nucleic acid is constructed using the given nucleic acid. For example, a cDNA or EST is derived from an expressed mRNA.

As used herein, the term “plant” includes whole plant, plant organ, plant tissues, and plant cell and progeny of same, but is not limited to angiosperms and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm and duckweed a well as fern and moss. Thus, a plant may be a monocot, a dicot, a vascular plant reproduced from spores such as fern or a nonvascular plant such as moss, liverwort, hornwort and algae. The term “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields.

As used herein, the term “dicot” refers to a flowering plant whose embryos have two seed leaves or cotyledons. Examples of dicots include Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus.

As used herein, the term “monocot” refers to a flowering plant whose embryos have one cotyledon or seed leaf. Examples of monocots include turf grass, maize (corn), rice, oat, wheat, barley, sorghum, orchid, iris, lily, onion, palm, and duckweed.

The term “seed” is meant to encompass the whole seed and/or all seed components, including, for example, the coleoptile and leaves, radicle and colorhiza, scutellum, starchy endosperm, aleurone layer, pericarp and/or testa, either during seed maturation and seed germination.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616 and 6,384,301, all of which are incorporated herein by reference in its entirety. Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682 and 6,603,061 and U.S. Patent Application publication US 2004/0216189 A1, all of which are incorporated herein by reference in its entirety.

The term “plant tissue” refers to any part of a plant, such as a plant organ. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro. As used herein, the term “tagged protein” refers to a recombinant protein that includes additional peptides that are not part of the native protein and that remain after post-translational processing.

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to ±10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, “percent (%) sequence identity” with respect to the nucleic acid or amino acid sequences identified herein is defined as the percentage of nucleic acid or amino acid residues in a candidate sequence that are identical with the amino acid residues in the polypeptide being compared, after aligning the sequences considering any conservative substitutions as part of the sequence identity.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. In some cases, a polynucleotide is exogenous (e.g. a heterologous polynucleotide). In some cases, a polynucleotide is endogenous to a cell. In some cases, a polynucleotide can exist in a cell-free environment. In some cases, a polynucleotide is a gene or fragment thereof. In some cases, a polynucleotide is DNA. In some cases, a polynucleotide is RNA. A polynucleotide can have any three-dimensional structure, and can perform any function, known or unknown. In some cases, a polynucleotide comprises one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), non-coding RNA, ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. In some cases, the sequence of nucleotides is interrupted by non-nucleotide components.

As used herein, the term “in vitro” is used to describe an event that takes place contained in a container for holding a laboratory reagent such that it is separated from the living biological source organism from which the material is obtained. In vitro assays can encompass cell-based assays in which live or dead cells or other biological materials are employed. In vitro assays can also encompass a cell-free assay in which no intact cells are employed.

As used herein, the term “in vivo” is used to describe an event that takes place in a plant, for example, inside a plant cell.

A “plasmid” as used herein, generally refers to a non-viral expression vector, e.g., a nucleic acid molecule that encodes for genes and/or regulatory elements necessary for the expression of genes. The term “vector,” as used herein, generally refers to a nucleic acid molecule capable of transferring or transporting a payload nucleic acid molecule. The payload nucleic acid molecule can be generally linked to, e.g., inserted into, the vector nucleic acid molecule. A vector can include sequences that direct autonomous replication in a cell, or can include sequences sufficient to allow integration into host cell gene (e.g., host cell DNA). Examples of a vector can include, but are not limited to, plasmids (e.g., DNA plasmids or RNA plasmids), transposons, cosmids, bacterial artificial chromosomes, and viral vectors. A vector of any of the aspects of the present disclosure can comprise exogenous, endogenous, or heterologous control sequences such as promoters and/or enhancers. For example, a “vector” can be a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used herein, the term “essentially free” means a component, if present, is present in an amount that does not contribute, or contributes only in a de minimus fashion, to the properties or function of the composition. In some cases, where a composition is essentially free of a particular component, the component is present in trace amounts, for example, less than 5% by weight, less than 4% by weight, less than 3% by weight, less than 2% by weight, less than 1% by weight, less than 0.5% by weight, less than 0.1% by weight, less than 0.05%, or less than 0.01% by weight. As used herein, “in trace amounts” means that the component is detectable using a testing method known to a person of ordinary skill in the field.

### EXAMPLES

The following illustrative examples are representative of embodiments of the compositions, systems, and methods described herein and are not meant to be limiting in any way.

### Example 1. Construction of Expression Plasmids for Plant Transformation

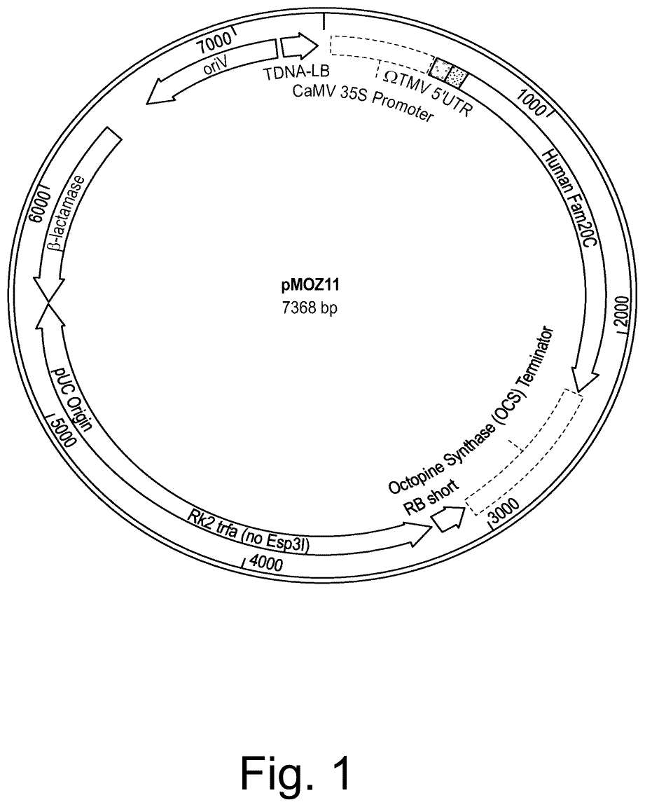

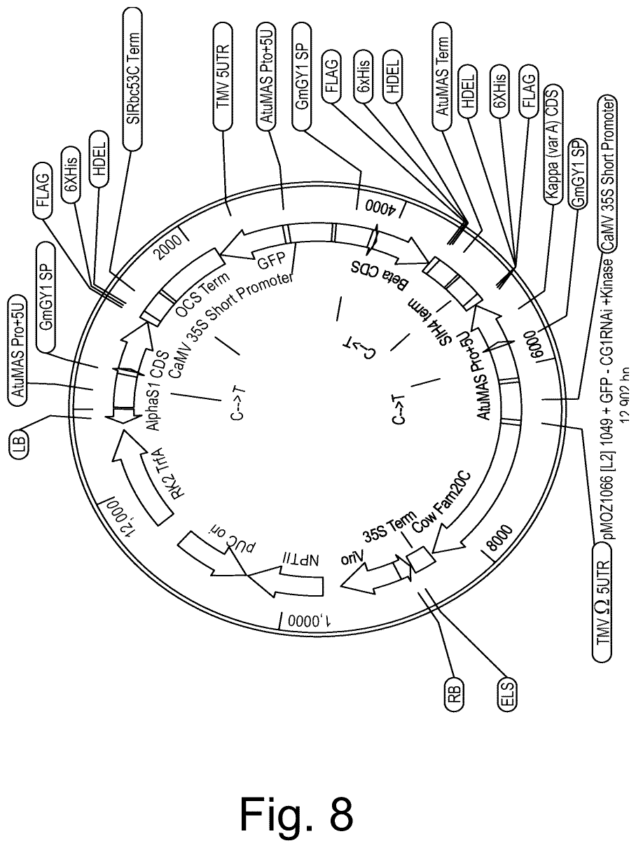

Referring now to FIG. 1, therein is shown an example of a pMOZ vector backbone [SEQ ID NO: 25], which is an example vector backbone used to construct expression plasmids for the production of casein and kinases in plants. As a specific example, FIG. 8 is a map of a vector backbone including a nucleic acid sequence encoding a β-lactamase conferring resistance to carbenicillin that allows for the plasmid to be selected for in E. coli, a first origin of replication (pUC ori) [SEQ ID NO:26] that allows for the plasmid to be propagated in E. coli, and a second origin of replication (oriV) [SEQ ID NO:27] that allows for the plasmid to be propagated in either E. coli or Agrobacterium. In this way, expression plasmids can be assembled using standard cloning methods in bacteria and then transferred to Agrobacterium for transformation into plants.

Continuing this example, the vector backbone further includes two Eco31I restriction sites that allow for cloning of a single expression cassette into the vector backbone using standard GoldenGate or MoClo methods, an identification nucleic acid sequence encoding the lacZ gene (lacZ) [SEQ ID NO:28] to aid in the identification of correct clones through E. coli colony blue/white screening. The Eco31I sites are flanked by a left border repeat (LB) and a right border repeat (RB) from nopaline C58 T-DNA [SEQ ID NO:29] that are recognized by Agrobacterium and allow for an expression cassette to be transformed into plant cells and integrated into the plant host genome.

Referring now to FIG. 1, therein is shown an example of an expression plasmid for the production of kinase proteins in plants constructed using the vector backbone using standard GoldenGate or MoClo methods. As a specific example, FIG. 1 is a map of a pMOZ11 expression plasmid, which is an example expression plasmid for the production of recombinant H. sapiens Fam20C in plants. Continuing this example, the pMOZI 1 expression plasmid includes a 1674 bp nucleic acid sequence encoding H. sapiens Fam20C (HsFam20C) [SEQ ID NO:31] that had been synthesized using only coding sequences from the original H. sapiens Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:7], a 62 bp translational enhancer encoding the omega leader sequence of the Tobacco Mosaic Virus (22 TMV 5UTR) [SEQ ID NO:23], a 57 bp nucleic acid sequence encoding the Arabidopsis thaliana ARA12 signal peptide gene (ARA12 Signal Peptide) [SEQ ID NO:36] for localizing the recombinant protein to the endoplasmic reticulum of the plant cell, and a 15 bp sequence coding for the endoplasmic reticulum retention peptide HDEL [SEQ ID NO: 30]. PMOZ11 was assembled using the enzyme Eco31I and standard GoldenGate protocols. The resulting plasmid was transformed into E. coli and later purified using a standard miniprep protocol. Sanger sequencing was used to verify the correct assembly of the plasmid.

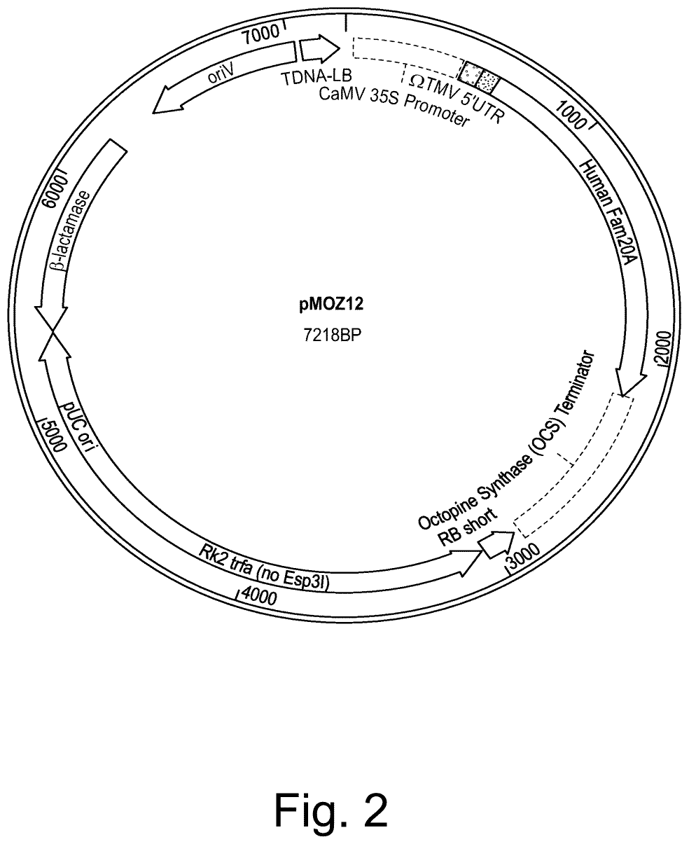

As another specific example, FIG. 2 is a map of a pMOZ12 expression plasmid, which is an example expression plasmid for the production of recombinant H. sapiens Fam20A in plants. Continuing this example, the pMOZ12 expression plasmid includes a 1524 bp nucleic acid sequence encoding H. sapiens Fam20A (HsFam20A) [SEQ ID NO:32] that had been synthesized using only coding sequences from the original H. sapiens Fam20C gene and was codon optimized for expression in plants using Nicotiana benthamiana as the model expression system, operatively linked to a 426bp sequence encoding the promoter Cauliflower Mosaic Virus 35S promoter (CaMV 35S Promoter) [SEQ ID NO:7], a 62 bp translational enhancer encoding the omega leader sequence of the Tobacco Mosaic Virus (22 TMV 5UTR) [SEQ ID NO:23], a 57 bp nucleic acid sequence encoding the Arabidopsis thaliana ARA12 signal peptide gene (ARA12 Signal Peptide) [SEQ ID NO:36] for localizing the recombinant protein to the endoplasmic reticulum of the plant cell, and a 15 bp sequence coding for the endoplasmic reticulum retention peptide HDEL [SEQ ID NO:30]. PMOZ12 was assembled using the enzyme Eco31I and standard GoldenGate protocols. The resulting plasmid was transformed into E. coli and later purified using a standard miniprep protocol. Sanger sequencing was used to verify the correct assembly of the plasmid.

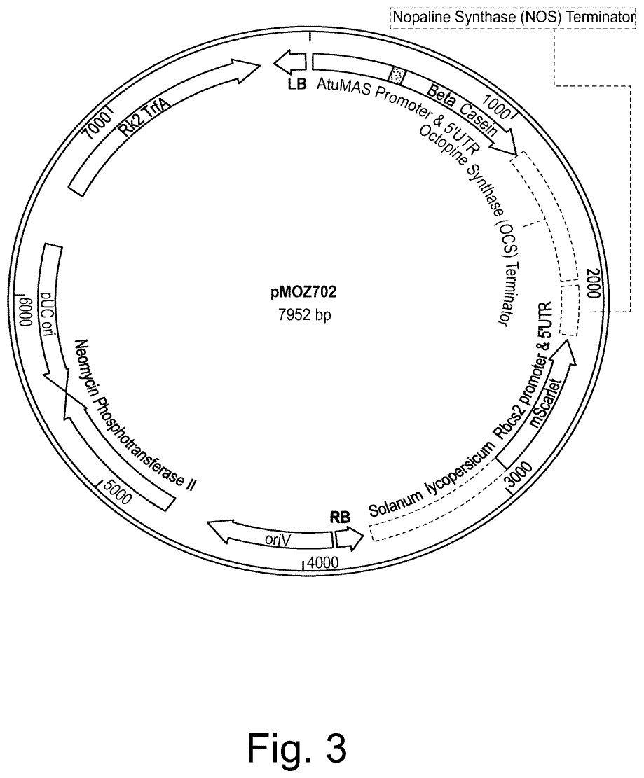

As another specific example, FIG. 3 is a map of a pMOZ702 expression plasmid, which is an example expression plasmid for the production of recombinant B. taurus Beta Casein in plants. Continuing this example, the pMOZ702 expression plasmid includes a 627 bp nucleic acid sequence encoding B. taurus β-casein [SEQ ID NO:42] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:35], a 63 bp nucleic acid sequence encoding the Arabidopsis thaliana S2S signal peptide (AtS2S Signal Peptide) [SEQ ID NO:37] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO: 38]. These sequences code for a protein containing a signal peptide, beta casein, HA tag, 6 histidine tag, and HDEL peptide [SEQ ID NO:46]. The plasmid also contains a 732 bp nucleic acid sequence coding for the mScarlet fluorescent protein [SEQ ID NO: 40] that had been synthesized and was codon optimized for expression in plants using Nicotiana benthamiana as the model expression system, operatively linked to a 739 bp sequence encoding the S. lycopersicum RbcS2 promoter (SIRbcS2 Promoter) [SEQ ID NO: 24], and a 263 bp sequence encoding the A. tumefaciens Nopaline Synthase Terminator (NOS Terminator) [SEQ ID NO: 41]. pMOZ702 was assembled using the enzyme Bpil and standard GoldenGate protocols. The resulting plasmid was transformed into E. coli and later purified using a standard miniprep protocol. Sanger sequencing was used to verify the correct assembly of the plasmid.

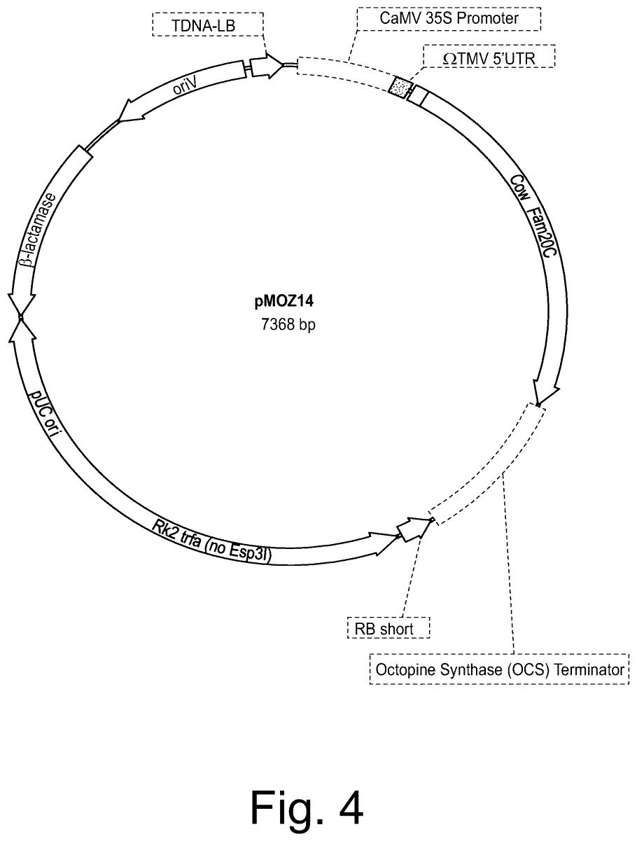

As another specific example, FIG. 4 is a map of a pMOZ14 expression plasmid, which is an example expression plasmid for the production of recombinant B. taurus Fam20C in plants. Continuing this example, the pMOZ14 expression plasmid includes a 1674 bp nucleic acid sequence encoding B. taurus Fam20C (BtFam20C) [SEQ ID NO:33] that had been synthesized using only coding sequences from the original B. taurus Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:7], a 62 bp translational enhancer encoding the omega leader sequence of the Tobacco Mosaic Virus (22 TMV 5UTR) [SEQ ID NO:23], a 57 bp nucleic acid sequence encoding the Arabidopsis thaliana ARA12 signal peptide gene (ARA12 Signal Peptide) [SEQ ID NO:36] for localizing the recombinant protein to the endoplasmic reticulum of the plant cell, and a 15 bp sequence coding for the endoplasmic reticulum retention peptide HDEL [SEQ ID NO:30]. PMOZ14 was assembled using the enzyme Eco311 and standard GoldenGate protocols. The resulting plasmid was transformed into E. coli and later purified using a standard miniprep protocol. Sanger sequencing was used to verify the correct assembly of the plasmid.

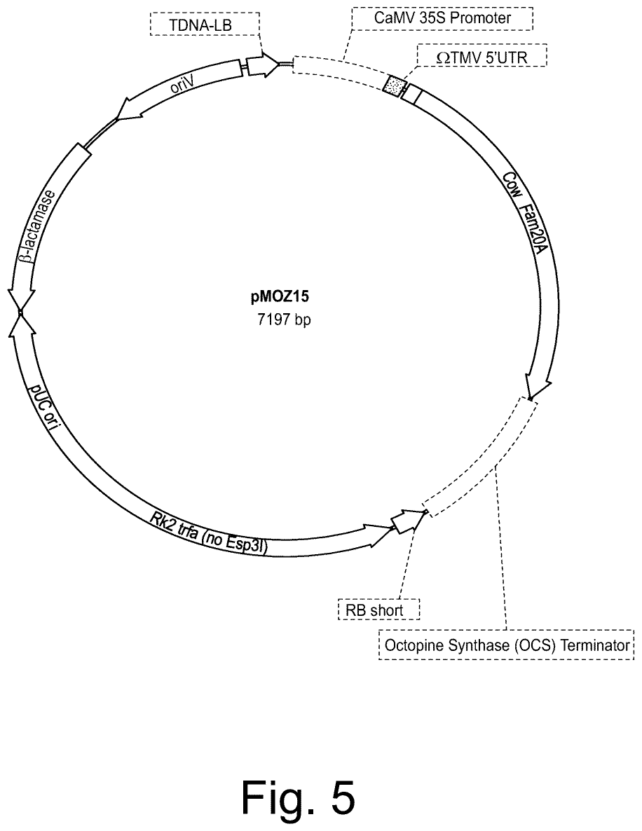

As another specific example, FIG. 5 is a map of a pMOZ15 expression plasmid, which is an example expression plasmid for the production of recombinant B. taurus Fam20A in plants. Continuing this example, the pMOZ15 expression plasmid includes a 1503 bp nucleic acid sequence encoding B. taurus Fam20A (BtFam20A) [SEQ ID NO:34] that had been synthesized using only coding sequences from the original B. taurus Fam20A gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:7], a 62 bp translational enhancer encoding the omega leader sequence of the Tobacco Mosaic Virus (22 TMV 5UTR) [SEQ ID NO:23], a 57 bp nucleic acid sequence encoding the Arabidopsis thaliana ARA12 signal peptide gene (ARA12 Signal Peptide) [SEQ ID NO:36] for localizing the recombinant protein to the endoplasmic reticulum of the plant cell, and a 15 bp sequence coding for the endoplasmic reticulum retention peptide HDEL [SEQ ID NO:30]. PMOZ15 was assembled using the enzyme Eco311 and standard GoldenGate protocols. The resulting plasmid was transformed into E. coli and later purified using a standard miniprep protocol. Sanger sequencing was used to verify the correct assembly of the plasmid.

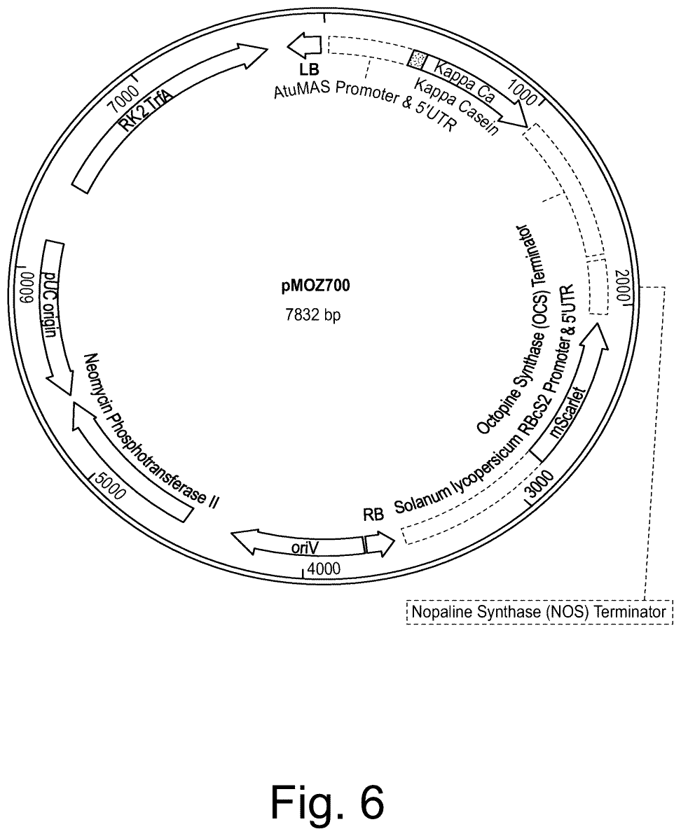

As another specific example, FIG. 6 is a map of a pMOZ700 expression plasmid, which is an example expression plasmid for the production of recombinant B. taurus Kappa Casein in plants. Continuing this example, the pMOZ700 expression plasmid includes a 507 bp nucleic acid sequence encoding B. taurus κ-casein [SEQ ID NO:43] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:35], a 63 bp nucleic acid sequence encoding the Arabidopsis thaliana S2S signal peptide (AtS2S Signal Peptide) [SEQ ID NO:37] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO: 38]. These sequences code for a protein containing a signal peptide, beta casein, HA tag, 6 histidine tag, and HDEL peptide [SEQ ID NO:47]. The plasmid also contains a 732 bp nucleic acid sequence coding for the mScarlet fluorescent protein [SEQ ID NO: 40] that had been synthesized and was codon optimized for expression in plants using Nicotiana benthamiana as the model expression system, operatively linked to a 739 bp sequence encoding the S. lycopersicum RbcS2 promoter (SIRbcS2 Promoter) [SEQ ID NO: 24], and a 263 bp sequence encoding the A. tumefaciens Nopaline Synthase Terminator (NOS Terminator) [SEQ ID NO: 41]. pMOZ702 was assembled using the enzyme Bpil and standard GoldenGate protocols. The resulting plasmid was transformed into E. coli and later purified using a standard miniprep protocol. Sanger sequencing was used to verify the correct assembly of the plasmid.

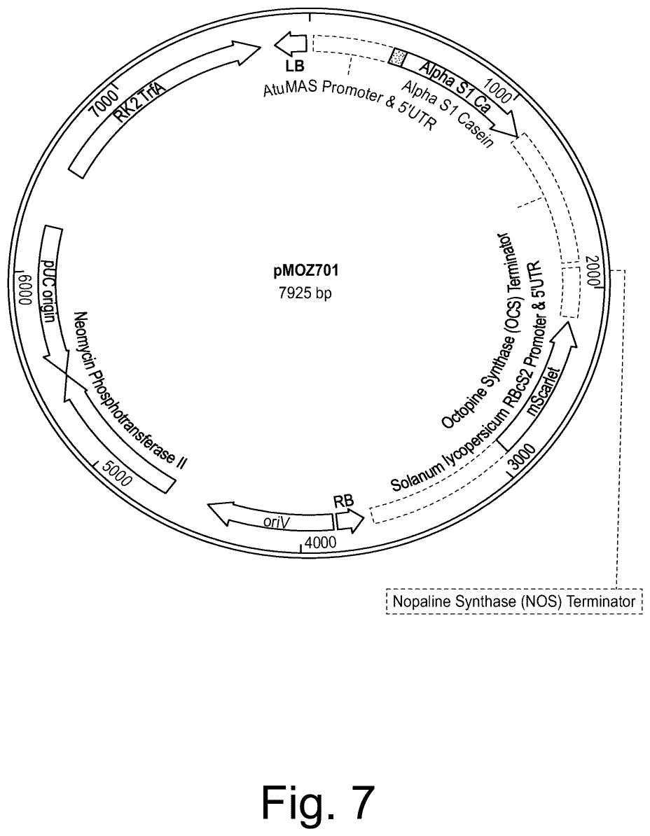

As another specific example, FIG. 7 is a map of a pMOZ701 expression plasmid, which is an example expression plasmid for the production of recombinant B. taurus αS1 Casein in plants. Continuing this example, the pMOZ701 expression plasmid includes a 597 bp nucleic acid sequence encoding B. taurus αS1-casein [SEQ ID NO:44] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:35], a 63 bp nucleic acid sequence encoding the Arabidopsis thaliana S2S signal peptide (AtS2S Signal Peptide) [SEQ ID NO:37] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO: 38]. These sequences code for a protein containing a signal peptide, beta casein, HA tag, 6 histidine tag, and HDEL peptide [SEQ ID NO:45]. The plasmid also contains a 732 bp nucleic acid sequence coding for the mScarlet fluorescent protein [SEQ ID NO: 40] that had been synthesized and was codon optimized for expression in plants using Nicotiana benthamiana as the model expression system, operatively linked to a 739 bp sequence encoding the S. lycopersicum RbcS2 promoter (SIRbcS2 Promoter) [SEQ ID NO: 24], and a 263 bp sequence encoding the A. tumefaciens Nopaline Synthase Terminator (NOS Terminator) [SEQ ID NO: 41]. pMOZ701 was assembled using the enzyme Bpil and standard GoldenGate protocols. The resulting plasmid was transformed into E. coli and later purified using a standard miniprep protocol. Sanger sequencing was used to verify the correct assembly of the plasmid.

Another specific example, FIG. 8 is a map of a pMOZ1066 expression plasmid, which is an example expression plasmid for the production of recombinant B. taurus aS1, Beta, and Kappa Casein, and Fam20C in plants. Continuing this example, the pMOZ1066 expression plasmid includes a 597 bp nucleic acid sequence encoding B. taurus αS1-casein [SEQ ID NO:44], a 627 bp nucleic acid sequence encoding B. taurus β-casein [SEQ ID NO:42], and a 507 bp nucleic acid sequence encoding B. taurus κ-casein [S EQ ID NO: 43] each being synthesized and codon optimized for expression in plants using Nicotiana benthamiana as the model expression system, each operatively linked to a 383 bp sequence encoding the promoter A. tumefaciens Mannopine Synthase promoter (AtuMAS Promoter) [SEQ ID NO:35], a GmGYI signal peptide, and an HDEL tag. Each casein is also tagged with a FLAG and 6× His. These sequences code for a protein containing a GY1 signal peptide, αS1-casein, FLAG tag, 6 histidine tag, and HDEL peptide [SEQ ID NO: 50], a protein containing a GY1 signal peptide, β-casein, FLAG tag, 6 histidine tag, and HDEL peptide [SEQ ID NO: 51], and a protein containing a GY1 signal peptide, κ-casein, FLAG tag, 6 histidine tag, and HDEL peptide [SEQ ID NO: 52]. The plasmid also contains a 1674 bp nucleic acid sequence encoding B. taurus Fam20C (BtFam20C) [SEQ ID NO:33], codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:7], and a 62 bp translational enhancer encoding the omega leader sequence of the Tobacco Mosaic Virus (22 TMV 5UTR) [SEQ ID NO:23]. PMOZ1066 was assembled using the enzyme Eco311 and standard GoldenGate protocols. The resulting plasmid was transformed into E. coli and later purified using a standard miniprep protocol. Sanger sequencing was used to verify the correct assembly of the plasmid.

### Example 2. Coexpression of 1 Casein and 2 Human Kinases

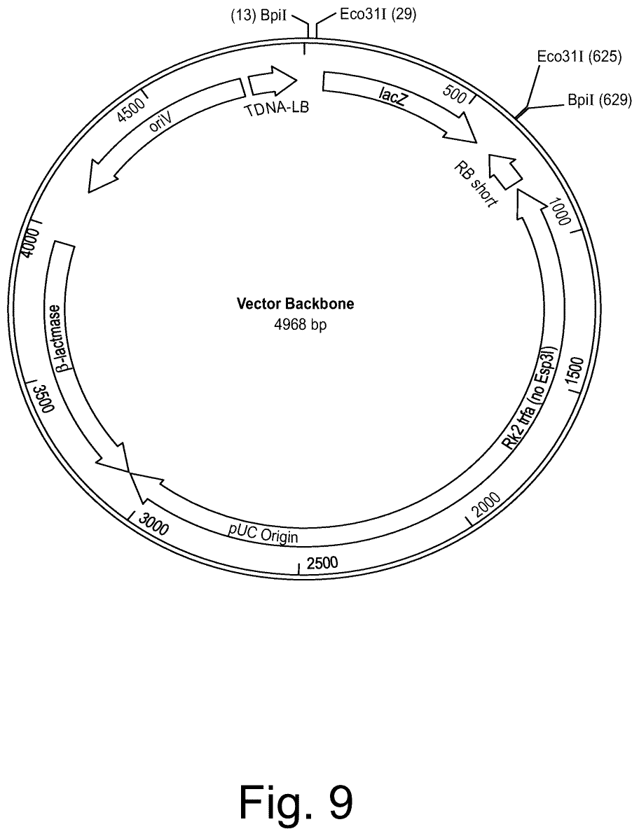

Referring to FIG. 9, therein is shown an example of the expression of recombinant Beta casein, HsFam20C, and HsFam20A in a systemically-infected N. benthamiana plant using combinations of the pMOZ11 (expresses HsFam20C), pMOZ12 (expresses HsFam20A) and pMOZ702 (expresses beta casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the plants were infiltrated with three different cultures of A. tumefaciens strain GV3101 carrying pMOZ702, pMOZ11, and pMOZ12 all grown to an OD600 of 0.1.

In a second condition the plants were infiltrated with three different cultures of A. tumefaciens strain GV3101 carrying pMOZ702, pMOZ11, and pMOZ12 all grown to an OD600 of 0.05.

In a third condition the plants were infiltrated with one culture of A. tumefaciens strain GV3101 carrying pMOZ702 grown to an OD600 of 0.1.

Following vacuum infiltration, plants were blot-dried and returned to the growth room for 72 hours before being imaged. Infected leaves were imaged with an epifluorescent microscope with Red Fluorescent Protein (RFP) excitation and emission filters to confirm that the mScarlet protein from pMOZ702 was successfully expressing in the plant cells. Leaves that were expressing mScarlet were harvested, frozen with liquid nitrogen, crushed with a mortar and pestle. 250 mg of crushed plant tissue was transferred to a 1.7mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL IM Trehalose, 3 mL diH2O) was added. This mixture was incubated on a rotisserie at 4C for 1 hour and then centrifuged at 400 RPM in with an Eppendorf 5415R centrifuge to pellet the solid plant material. The supernatant containing the extracted protein was transferred to a new 1.7 mL tube.

Further continuing this example, protein samples from the infected plant tissue were analyzed with sodium dodecyl sulfate polyacrylamide gel electrophoresis (SDS PAGE). 10μL of supernatant was mixed with 30μL of protein loading buffer (900 μL of 4× Laemmli Sample Buffer [Bio-Rad Laboratories]+100 μL of 2-mercaptoethanol) and heated for 5 minutes at 95C. These samples were loaded onto Bio-Rad “Any kD” precast polyacrylamide gels along with a standard protein ladder and phosphorylated and dephosphorylated beta casein samples from Sigma Aldrich. The gel was run in 1× Tris/Glycine/SDS Buffer (Bio-Rad Laboratories) at 150V for 45 minutes. The gel was removed from the gel box and placed in a PVDF Transfer Pack (Bio-Rad Laboratories), the transfer pack was placed in a Trans-Blot Turbo (Bio-Rad Laboratories) and the proteins were transferred to the PVDF membrane using the “Mini TGX” settings. The PVDF membrane containing the transferred proteins was first washed in 25 mL Protein Free Blocking Buffer (ThermoFisher) for 1 hour, then incubated with 5 mL of Protein Free Blocking Buffer containing 5 μL of anti-beta-casein polyclonal rabbit IgG at room temperature for 4 hours. The membrane was then washed three times with 10 mL TBST (20 mM Tris-HCl, 150 mM NaCl, 0.1% Tween 20) for 10 minutes at room temperature. Then the membrane was washed with 25 mL of Protein Free Blocking Buffer containing 2 μL of anti-rabbit IgG secondary antibody conjugated to horseradish peroxidase for 1 hour at room temperature and then poured off. The membrane was placed in a ChemiDoc MP imaging system (Bio-Rad Laboratories) and ImL of SuperSignal West Pico (ThermoFisher) luminescent imaging solution was added to the membrane. Images were captured using the Chemiluminescence setting on the ChemiDoc MP.

Further continuing this example, the casein proteins expressed in plant cells show up on the anti-beta-casein Western blot with varying migration distances (FIG. 9). Lane 1 purified beta casein standard. Lane 2: dephosphorylated beta casein standard. Lane 3: Tobacco leaf tissue transformed with pMOZ702+pMOZ11+pMOZ12. Lane 4: Tobacco leaf tissue transformed with pMOZ702+pMOZ11+pMOZ12 using half concentration of A. tumefaciens. Lane 5: Tobacco leaf tissue transformed with only pMOZ702. In lanes containing beta casein coexpressed with 2 human kinases the bands are shifted upward on the gel relative to the sample transformed with only beta casein, suggesting that the molecular weight of the beta casein has increased due to phosphorylation.

### Example 3. Coexpression of 1 Casein and 1 or 2 Kinases

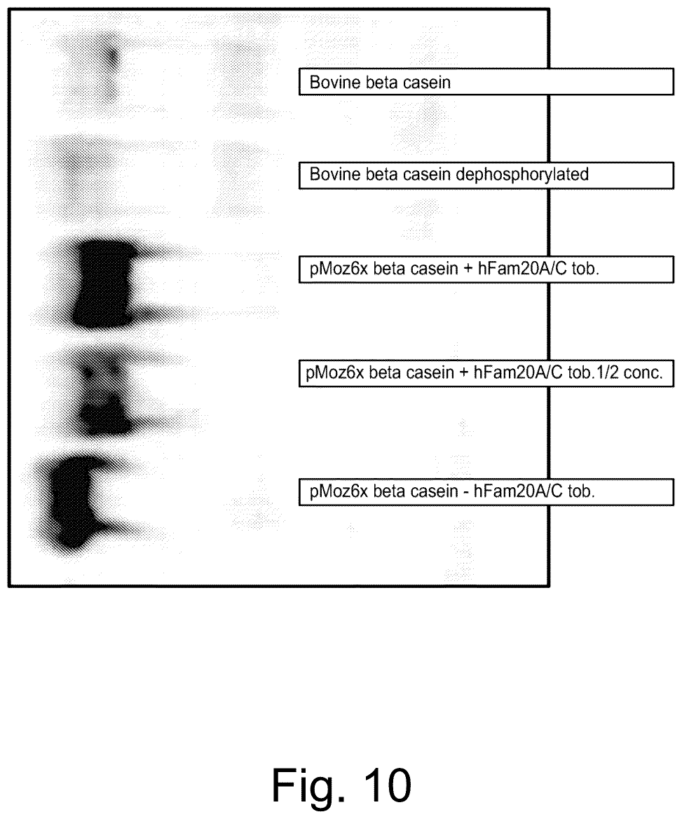

Referring to FIG. 10, therein is shown an example of the expression of recombinant Beta casein, HsFam20C, and HsFam20A in a systemically-infected N. benthamiana plant using combinations of the pMOZ11 (expresses HsFam20C), pMOZ12 (expresses HsFam20A), pMOZ14 (expresses BtFam20C), pMOZ15 (expresses BtFam20A) and pMOZ702 (expresses beta casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids at an OD600 of 0.1.

In one condition the plants were infiltrated with two different cultures of A. tumefaciens strain GV3101 carrying pMOZ702, pMOZ11.

In a second condition the plants were infiltrated with three different cultures of A. tumefaciens strain GV3101 carrying pMOZ702, pMOZ11, and pMOZ12.

In a third condition the plants were infiltrated with a single culture of A. tumefaciens strain GV3101 carrying pMOZ702.

In a fourth condition the plants were infiltrated with two different cultures of A. tumefaciens strain GV3101 carrying pMOZ702, pMOZ14.

In a fifth condition the plants were infiltrated with three different cultures of A. tumefaciens strain GV3101 carrying pMOZ702, pMOZ14, and pMOZ15.

Following vacuum infiltration, plants were blot-dried and returned to the growth room for 72 hours before being imaged. Infected leaves were imaged with an epifluorescent microscope with Red Fluorescent Protein (RFP) excitation and emission filters to confirm that the mScarlet protein from pMOZ702 was successfully expressing in the plant cells. Leaves that were expressing mScarlet were harvested, frozen with liquid nitrogen, crushed with a mortar and pestle. 250 mg of crushed plant tissue was transferred to a 1.7 mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL 1 M Trehalose, 3 mL diH2O) was added. This mixture was incubated on a rotisserie at 4C for 1 hour and then centrifuged at 400 RPM in with an Eppendorf 5415R centrifuge to pellet the solid plant material. The supernatant containing the extracted protein was transferred to a new 1.7 mL tube.

Further continuing this example, protein samples from the infected plant tissue were analyzed with sodium dodecyl sulfate polyacrylamide gel electrophoresis (SDS PAGE). 10 μL of supernatant was mixed with 30 μL of protein loading buffer (900 μL of 4× Laemmli Sample Buffer [Bio-Rad Laboratories]+100 μL of 2-mercaptoethanol) and heated for 5 minutes at 95C. These samples were loaded onto Bio-Rad “Any kD” precast polyacrylamide gels along with a standard protein ladder and phosphorylated and dephosphorylated beta casein samples from Sigma Aldrich. The gel was run in 1× Tris/Glycine/SDS Buffer (Bio-Rad Laboratories) at 150V for 45 minutes. The gel was removed from the gel box and placed in a PVDF Transfer Pack (Bio-Rad Laboratories), the transfer pack was placed in a Trans-Blot Turbo (Bio-Rad Laboratories) and the proteins were transferred to the PVDF membrane using the “Mini TGX” settings. The PVDF membrane containing the transferred proteins was first washed in 25 mL Protein Free Blocking Buffer (ThermoFisher) for 1 hour, then incubated with 5 mL of Protein Free Blocking Buffer containing 5 μL of anti-beta-casein polyclonal rabbit IgG at room temperature for 4 hours. The membrane was then washed three times with 10 mL TBST (20 mM Tris-HCl, 150 mM NaCl, 0.1% Tween 20) for 10 minutes at room temperature. Then the membrane was washed with 25 mL of Protein Free Blocking Buffer containing 2 μL of anti-rabbit IgG secondary antibody conjugated to horseradish peroxidase for 1 hour at room temperature and then poured off. The membrane was placed in a ChemiDoc MP imaging system (Bio-Rad Laboratories) and 1 mL of SuperSignal West Pico (ThermoFisher) luminescent imaging solution was added to the membrane. Images were captured using the Chemiluminescence setting on the ChemiDoc MP.

Further continuing this example, the casein proteins expressed in plant cells show up on the anti-beta-casein Western blot with varying migration distances (FIG. 10). Lane 1: Molecular weight ladder. Lane 2: Empty. Lane 3: Purified beta casein standard. Lane 4: dephosphorylated beta casein standard. Lane 5: Tobacco leaf tissue transformed with pMOZ702 and pMOZ11. Lane 6: Tobacco leaf tissue transformed with pMOZ702 and pMOZ14. Lane 5: Tobacco leaf tissue transformed with pMOZ702, pMOZ14, and pMOZ15. Lane 6: Tobacco leaf tissue transformed with pMOZ702, pMOZ11, and pMOZ12. Lane 5: Tobacco leaf tissue transformed with only pMOZ702. In lanes containing beta casein coexpressed with 1 human kinase, 2 human kinases, 1 bovine kinase or 2 bovine kinases the bands are shifted upward on the gel relative to the sample transformed with only beta casein, suggesting that the molecular weight of the beta casein has increased due to phosphorylation.

### Example 4. Phosphorylation Increased Casein Expression

In this proposed experiment the expression level of casein proteins is shown to increase when phosphorylated. Recombinant Kappa casein and BtFam20C kinase are expressed in a systemically-infected N. benthamiana plant using combinations of the pMOZ14 (expresses BtFam20C) and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ700, pMOZ14 all grown to an OD600 of 0.1.

In a second condition N. benthamiana plants were infiltrated with a single culture of A. tumefaciens strain GV3101 carrying pMOZ700 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL IM Trehalose, 3 mL diH2O) will be added. This mixture will be incubated on a rotisserie at 4C for 1 hour and then centrifuged to pellet the solid plant material. The supernatant containing the extracted protein will be transferred to a new tube.

Continuing this example, extracted protein will be analyzed with SDS PAGE followed by a Western blot with kappa casein antibodies using a typical Western blotting protocol. After incubating with an appropriate secondary antibody conjugated to horseradish peroxidase, luminescent developing solution such as SuperSignal West (ThermoFisher) will be applied to the Western membrane and imaged on a ChemiDoc MP (Bio-Rad Laboratories) imaging system. The brightness of the kappa casein bands will be quantified using functions built into the ChemiDoc MP. Brighter bands in lanes containing protein from the plants transformed with both pMOZ700 (Kappa casein) and pMOZ14 (kinase) compared to protein from plants transformed with only pMOZ700 (kappa casein) shows that phosphorylated casein proteins are expressed at higher concentrations than non-phosphorylated casein.

In a similar experiment, N. benthamiana plants will be transformed with the same casein and kinase plasmids and protein will be extracted the same as just described. Extracted protein supernatants will be analyzed by high pressure liquid chromatography (HPLC). The supernatants will be diluted with acetate buffer and loaded into the HPLC apparatus. Eluted protein will be detected and quantified by UV absorption. Integrals will be calculated for the peaks corresponding to casein proteins to quantify the concentration of casein in each sample. Larger integral values for casein proteins from plants transformed with casein and kinase compared to casein from plants transformed with only casein will show that phosphorylation of casein increases their expression level.

### Example 5. Phosphorylation Increased Aggregation of Casein Proteins

In this proposed experiment the aggregation of multiple caseins proteins is shown to be increased when the caseins are phosphorylated compared to non-phosphorylated caseins. Recombinant bovine alpha S1 casein, bovine beta casein and BtFam20C kinase will be expressed in a in a systemically-infected N. benthamiana plant using combinations of the pMOZ14 (expresses BtFam20C), pMOZ701 (expresses bovine alpha SI casein), and pMOZ702 (expresses bovine beta casein) expression plasmids. In this example, N. benthamiana plants will be incubated in a growth room at 25° C. with a 16 hour light 8 hour dark cycle for 4 weeks. The 4-week old N. benthamiana plants will be infiltrated with A. tumefaciens strain GV3101 carrying combinations of pMOZ plasmids.

In one condition the N. benthamiana plants were infiltrated with two different cultures of A. tumefaciens strain GV3101 carrying pMOZ701 and pMOZ14 all grown to an OD600 of 0.1.

In a second condition the N. benthamiana plants were infiltrated with two different cultures of A. tumefaciens strain GV3101 carrying pMOZ702 and pMOZ14 all grown to an OD600 of 0.1.

In a third condition the N. benthamiana plants were infiltrated with a single culture of A. tumefaciens strain GV3101 carrying pMOZ701 grown to an OD600 of 0.1.

In a fourth condition the N. benthamiana plants were infiltrated with a single culture of A. tumefaciens strain GV3101 carrying pMOZ702 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ701 or pMOZ702 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL IM Trehalose, 3 mL diH2O) will be added. This mixture will be incubated on a rotisserie at 4C for 1 hour and then centrifuged to pellet the solid plant material. The supernatant containing the extracted protein will be transferred to a new tube for further analysis.

Continuing this example, various combinations of protein supernatants from the different transformation conditions will be used in a co-immunoprecipitation (CoIP) assay to measure the amount of protein-protein aggregation. In each CoIP assay protein supernatant from one sample will be mixed with magnetic anti-HA Dynabeads (ThermoFisher catalog #88837) so that the HA peptide tag attached to the casein protein expressed from either pMOZ701 or pMOZ702 plasmid is contacted with anti-HA antibodies attached to the magnetic beads. The quantity of protein added will be great enough to saturate all available HA antibodies on the surface of the beads. The HA-labeled casein proteins will stick to the magnetic beads and the rest of the supernatant will be washed away with wash buffer ((10 mM Tris pH 7.4, 1 mM EDTA, 1 mM EGTA, pH 8.0, 150 mM NaCl, 1% Triton X-100, 0.2 mM sodium orthovanadate) while the casein is retained by the magnetic beads held stationary by an external magnetic force. Protein supernatant from a second sample will then be contacted with the beads and allowed to incubate at room temperature for 1 hour. The supernatant will then be washed with wash buffer while the beads are held stationary by an external magnetic force. Any protein stuck the beads will then be released from the beads by adding 30 μL of Laemmli buffer (65.8 mM Tris-HCl, pH 6.8, 2.1% SDS, 26.3% glycerol, 2% 2-mercaptocthanol) and incubating at 90 degrees celsius for 20 minutes. The beads will then be removed from the Laemmli buffer using a magnet and the remaining liquid will be analyzed by SDS PAGE and Western blot using standard protocols. The Western blot will be developed using a casein-specific antibody targeting the second casein protein that was added to the CoIP assay. Analyzing the brightness of the bands on the Western blot will show which samples captured more secondary casein protein.

In one condition, the supernatant from plants transformed with pMOZ701 will be first contacted with the beads and then supernatant from plants transformed with pMOZ702 will be contacted second.

In a second condition, the supernatant from plants transformed with pMOZ702 will be first contacted with the beads and then supernatant from plants transformed with pMOZ701 will be contacted second.

In a third condition, the supernatant from plants transformed with pMOZ702 and pMOZ14 will be first contacted with the beads and then supernatant from plants transformed with pMOZ701and pMOZ14 will be contacted second.

In a third condition, the supernatant from plants transformed with pMOZ701 and pMOZ14 will be first contacted with the beads and then supernatant from plants transformed with pMOZ702 and pMOZ14 will be contacted second.

Further continuing this example, Western blots showing increased amounts of casein eluted from the beads from samples where both casein plasmids (pMOZ701 or pMOZ702) were co-transformed with kinase plasmids (pMOZ14) compared to samples where the casein plasmids were transformed without kinase plasmid will indicate that phosphorylation of caseins increases their ability to aggregate or bind to each other.

### Example 6. Phosphorylation Improves Casein Micelle Formation

In this proposed experiment micelles will form in vivo when casein is phosphorylated. Recombinant Beta casein, Kappa casein, Alpha SI casein, and BtFam20C kinase are expressed in a in a systemically-infected N. benthamiana plant using combinations of the pMOZ14 (expresses BtFam20C), pMOZ702 (expresses bovine beta casein), pMOZ701 (expresses alpha casein), and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with four different cultures of A. tumefaciens strain GV3101 carrying pMOZ700, pMOZ701, pMOZ702, and pMOZ14 all grown to an OD600 of 0.1.

In a second condition N. benthamiana plants were infiltrated with three different cultures of A. tumefaciens strain GV3101 carrying pMOZ700, pMOZ701, and pMOZ702 all grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 or pMOZ701 or pMOZ702 were successfully expressed in the plant cells. Leaves that express mScarlet will be cut from the plant and then fixed in formaldehyde and osmium tetroxide using standard fixation and clearing protocols. The fixed tissue will then be sectioned and imaged on a transmission electron microscope. Comparison of images of leaves transformed with and without pMOZ14 will show that casein micelles form when kinase is present to phosphorylate the casein protein.

### Example 7. Phosphorylation Increased Viscosity, Quality or Mouthfeel

In this proposed experiment the amount of calcium bound to casein proteins is shown to be increased when casein is phosphorylated. Recombinant Kappa casein and BtFam20C kinase are expressed in a in a systemically-infected N. benthamiana plant using combinations of the pMOZ14 (expresses BtFam20C) and pMOZ702 (expresses bovine alpha casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ702, pMOZ14 all grown to an OD600 of 0.1.

In a second condition N. benthamiana plants were infiltrated with a single culture of A. tumefaciens strain GV3101 carrying pMOZ702 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL IM Trehalose, 3 mL diH2O) will be added. This mixture will be incubated on a rotisserie at 4C for 1 hour and then centrifuged to pellet the solid plant material. The supernatant containing the extracted protein will be transferred to a new tube.

Continuing this example, the viscosity of protein supernatants of samples transformed with and without the kinase encoded by pMOZ14 will be measured and compared. 10 μL of each supernatant will be loaded into RheoSense microVISC viscometer and the viscosities will be measured. Results showing higher values for samples co-transformed with pMOZ14 indicate that phosphorylation of caseins increases viscosity of solutions containing those casein proteins.

### Example 8. Phosphorylated Casein Increased Calcium Binding

In this proposed experiment the amount of calcium bound to casein proteins is shown to be increased when casein is phosphorylated. Recombinant Kappa casein and BtFam20C kinase arc expressed in a in a systemically-infected N. benthamiana plant using combinations of the pMOZ14 (expresses BtFam20C) and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ700, pMOZ14 all grown to an OD600 of 0.1.

In a second condition N. benthamiana plants were infiltrated with a single culture of A. tumefaciens strain GV3101 carrying pMOZ700 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 μL of protein extraction buffer (800 μL of 500 mM sodium phosphate, 200 μL of 500 mM sodium phosphate dibasic, 1 mL 200 mM Sodium metabisulfite, 50 μL Tween-20, 5 mL 1M Trehalose, 3 mL diH2O) will be added. This mixture will be incubated on a rotisserie at 4C for 1 hour and then centrifuged to pellet the solid plant material. The supernatant containing the extracted protein will be transferred to a new tube.

Continuing this example, protein supernatants will be assayed for calcium content by first purifying the protein using anti-HA magnetic beads, then by a colorimetric assay specific for calcium. Supernatant from either plants transformed with both pMOZ702 and pMOZ14 or plants only transformed with pMOZ702 will be mixed with magnetic anti-HA Dynabeads (ThermoFisher catalog #88837) so that the HA peptide tag attached to the casein protein expressed from either pMOZ702 plasmid is contacted with anti-HA antibodies attached to the magnetic beads. The HA-labeled casein proteins will stick to the magnetic beads and the rest of the supernatant will be washed away with wash buffer ((10 mM Tris pH 7.4. ImM EDTA, ImM EGTA, pH 8.0, 150 mM NaCl, 1% Triton X-100, 0.2 mM sodium orthovanadate) while the casein is retained by the magnetic beads held stationary by an external magnetic force. Any protein stuck to the beads will then be released from the beads by adding 30 μLTris buffer (10 mM Tris-HCl, pH 7.5) and incubating at 90 degrees celsius for 20 minutes. The beads will then be removed from the Tris buffer using a magnet. The remaining liquid will be assayed for calcium concentration using a Calcium Assay Kit from Abcam (catalog #ab102505). The protocol provided with the kit will be followed and the color intensity will be read out by a colorimetric plate reader. The values will be compared to a standard curve to calculate calcium concentrations. Results showing increased calcium in samples where both pMOZ702 and pMOZ14 were transformed compared to samples where only pMOZ702 was transformed will indicate that phosphorylated caseins bind calcium to a greater degree than non-phosphorylated caseins.

### Example 9. Casein Phosphorylation In Vivo

In this experiment, pMOZ1066 was transiently transformed into soybeans and fresh milk was produced in the plants. After the transformation, the soybeans were washed, incubated on plates, frozen in liquid nitrogen and then crushed into a powder. A protein extraction buffer (50 mM Tris, 300 mM KCl, 0.5% Tween-20, 3.65% glycerol, Sigma plant protease inhibitor, pH 8.6) was added to the crushed beans and the tissue was rotated at 4° C. for one hour, before it was centrifuged at 13,000 rpm for 1 hour at 4° C. The supernatant was then purified by nickel-affinity resin. A Ni-NTA resin agarose was mixed by vortex and 500 μL of resin slurry was placed into a new tube and centrifuged at 700×g for 2 minutes. The supernatant was discarded and 500 μL of equilibration buffer was added (50 mM Tris base, 300 mM KCl, 10 mM imidazole, pH to 7.4). The tube was then mixed and centrifuged at 1000× g for 2 minutes. This step was then repeated an additional time and the resin was kept in the buffer until use for purification. The soybean protein sample was then clarified using a 0.45 μm syringe filter, and the filtered sample was added to the resin tube and rotated for 2-4 hours at 4° C. The sample was then centrifuged for 2 minutes at 1000× g and the supernatant was then removed and 500 μL of wash buffer (50 mM Tris base, 300 mM KCl, 20 mM imidazole, pH to 7.4) was added. This centrifuge and wash step was repeated 4×. Next, 750 μL of elution buffer was added to the sample and the tube was rotated for 15 minutes at 4° C. The tube was then centrifuged for 2 minutes at 700×g. This elution and centrifugation step was repeated 4×. After purification, the resulting elution was concentrated in an Amicon concentrator and then dialyzed against water to decrease the concentration of imidazole and other reagents.

Continuing this experiment, “1066 milk” was made using this purified protein. 1 mL of the purified casein containing water was added to a 5 mL beaker and a mini stir bar was placed into the beaker. The 5 mL beaker was then placed inside of a 600 mL beaker on a hot plate containing approximately 125 ml of water to submerge the 5 ml beaker about a third of the height, using a clamping system. A thermometer was placed in the outer water with the bulb not touching the glass but fully submerged in the water. The hotplate was set to about 65C, and the casein was stirred until fully in solution. The stir bar was set to 1000 RPM. Next, 20 μL of tripotassium citrate and 70 μL potassium phosphate was added to the beaker and allowed to stir for 4 minutes. Then, every 4 minutes 12.5 μL of Potassium Phosphate Sol and 25 μL of Calcium Chloride Sol was added (a total of 12×). The solution was then stirred at 1000 RPM for 1 hour with the heat turned off, and 240 μL of water and 180 μL of heavy cream added.

Further continuing this experiment, “1066 cheese” was made using the “1066 milk” previously created. First, the milk was placed in an ice bath and the temperature was decreased to between about 8°-14° C. Next, 100 mg/mL of citric acid was added in 5 μL increments while stirring. The 5 mL beaker containing the milk was then removed from the larger beaker and the pH was checked (can be about pH 5.6). The 5 mL beaker was then resubmerged in the water containing 600 mL beaker and the hotplate was set to 120° C. with the stir bar set to 1000 RPM. Once the water in the outer beaker reached 32° C. 4 μL of Rennet solution was added to the milk. The Rennet/milk solution was stirred and then the stir bar was removed. The solution was allowed to sit for 20 minutes with the water temperature in the outer beaker at about 37° C. Once curds began to form, the small beaker was removed, and the curds were cut with a spatula in a criss-cross shape. The small beaker was then returned to the water bath and the water was heated to 41° C. Once the water reached 41° C. the small beaker was removed and covered with a cheesecloth after dumping off the whey. 3 mL of 80° C. water was added to the cheese and poured off after 5 minutes. Then, another 3 mL of 80° C. water was added and poured off after five minutes, after which the cheese was formed.

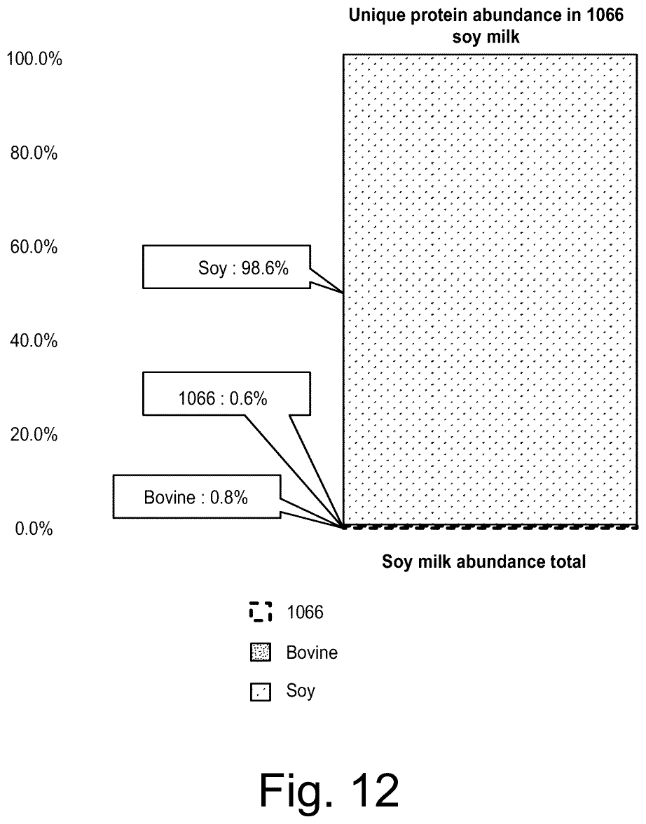

The 1066 milk was then submitted for mass spectrometry analysis, along with a sample of raw cow milk for qualitative comparison. The data was analyzed using the following method. First, contaminants were removed from the dataset (human hair, human skin). Proteins that were only detected by 1 peptide were removed, and proteins that were only detected by 1 unique peptide with less than 20% coverage of the protein sequence were removed. Finally, the protein abundances in each sample were normalized by summing each sample's abundances, and adjusting each sample total abundance to 1 by dividing individual abundances by the sum. This normalization allowed for the relative quantification of proteins within the 1066 milk. Two 1066 replicates were averaged, and two raw milk replicates were averaged. The 1066 milk was affinity purified, and the relative abundance of milk are shown in FIG. 12. FIG. 12 shows a unique protein abundance profile in 1066 milk. In purified “1066 milk”, soy proteins comprise over 95% total protein content, bovine proteins (including bovine casein proteins and the bovine kinase protein) is about 0.8% of total protein content (by weight). The 1066 protein (i.e., green fluorescent protein) is approximately 0.6% of total protein content (by weight).

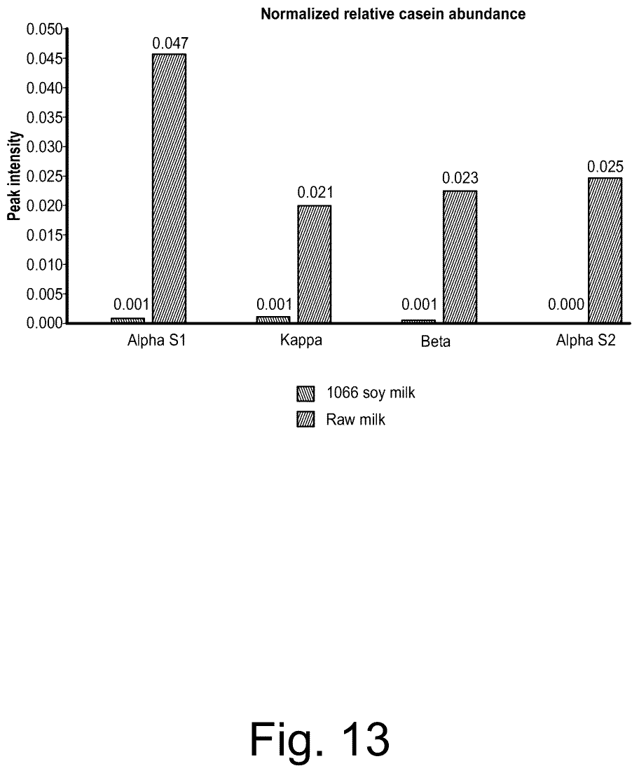

The relative abundances of casein in 1066 milk and in cow milk were measured with the results shown in FIG. 13. FIG. 13 shows normalized relative casein abundance in both 1066 milk and raw milk. While the amount of each casein protein in 1066 milk is lower compared to raw milk, all three casein types expressed in the plasmid are detected in the sample.

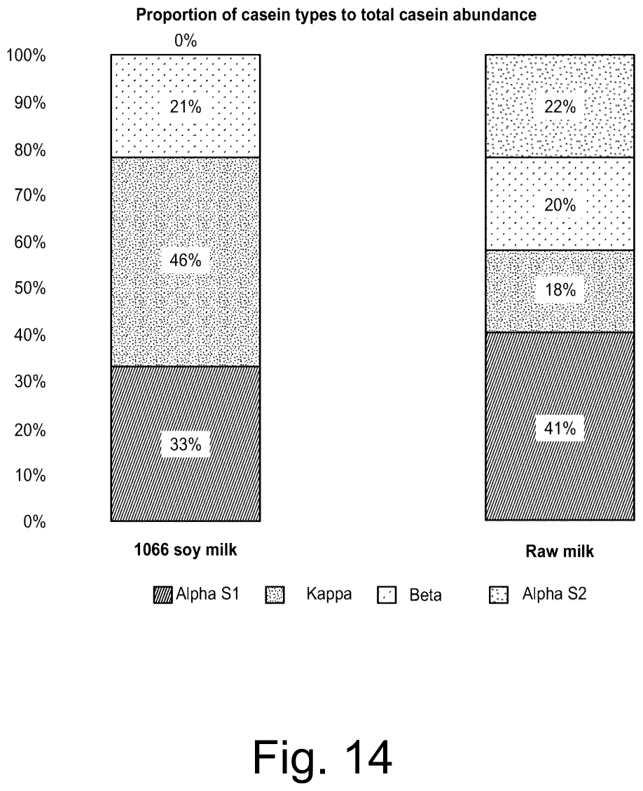

The proportion of each casein in the milks were compared, with the results shown in FIG. 14. FIG. 14 shows the proportion of each casein type out of the total casein amount in 1066 milk and in raw milk. The main difference between the 1066 milk and raw milk is the large amount of kappa casein found in 1066 milk (about 46% casein protein abundance) compared to raw milk (about 18% casein protein abundance). Alpha S1 casein protein accounts for about 33% casein protein abundance in 1066 milk, and about 41% casein protein abundance in raw milk. Alpha S2 accounts for about 22% casein protein abundance in raw milk, but is not detectable in 1066 milk.

To detect the phosphorylation in the casein proteins, a phosphopeptide enrichment was performed on the raw milk, but not the 1066 milk. The phosphopeptide enrichment was performed using the following protocol:
a. The protein disulfide bonds were reduced and alkylated by treatment with 10 mM tris (2-carboxyethyl) phosphine (TCEP) and 40 mM chloroacetamide (CAA) for 10 min at 950C (Add a tenth of the original volume of 110 mM (TCEP), 440 mM CAA in 50 mM mM triethyl ammonium bicarbonate (TEAB).
b. The protein solutions were then diluted five-fold with 50 mM TEAB.
c. Trypsin was added (ratio=1:100, mass trypsin: mass total protein) after being prepared in 50 mM TEAB and incubated at 37 C, while rotating for 3-4 hours.
d. Step c was repeated but the incubation was performed overnight.
e. Ethyl acetate+1% trifluoroacetic acid (TFA) was added (ratio=1:1, volume sample: volume ethyl acetate) and the samples were vortexed for 5 minutes.
f. The samples were centrifuged at 16,000× g for 5 minutes at room temperature and the supernatant was discarded (ethyl acetate).
g. The samples were then lyophilized in a centrifugal vacuum concentrator.
h. The samples were reconstituted in 2% acetonitrile+0.1% TFA and desalted on StageTips. The samples were then and lyophilized in a centrifugal vacuum concentrator.
i. The samples were reconstituted in 50 mM TEAB and a bicinchoninic acid peptide quantitation assay was performed (Thermo Fisher Scientific) as per manufacturer's protocol using a spectrophotometer/plate reader.
j. The samples were individually labeled using a TMT Isobaric Label Reagent Set (Thermo Fisher Scientific) according to manufacturer's protocol and then lyophilized in the centrifugal vacuum concentrator.
k. The samples were then reconstituted in 2% acetonitrile+0.1% TFA and combined with an equal small aliquot of each sample into a new microcentrifuge tube.
l. The samples were then desalted and lyophilized as in step h.
m. The samples were reconstituted in 2% acetonitrile +0.1% Formic Acid (FA), transferred to an autosampler vial, and analyzed by LC/MS² using a nanoLC and orbitrap mass analyzer using a C18 column and DDA/MS² mass spectrometer method.
n. The raw data was submitted to a database search using Proteome Discoverer. The results were checked for TMT labeling efficiency and sample volumes were adjusted as necessary to achieve an equal ratio of protein across the TMT labeled samples (Abundance Ratio=1).
o. The remaining samples were pooled in equal ratio and desalted/lyophilized as in step h.
p. Phosphorylated peptides were extracted using High-Select TiO2 and Fe-NTA kits (Thermo Fisher Scientific) as per manufacturer's protocol (flow through was kept for global proteomic analysis).
q. The flow through (global) and phophopeptide enriched samples, was fractionated separately, using Pierce High pH Reversed-Phase Kit according to manufacturer's protocol.
r. The fractions were lyophilized in the centrifugal vacuum concentrator and reconstituted in 2% acetonitrile+0.1% TFA.

s. The samples were transferred to autosampler vials and LC/MS² was performed as in step m.
t. All raw data was submitted to a single database search using Proteome Discoverer.

Following the phosphopeptide enrichment of the raw milk, the samples were analyzed by LC/MS² using a nanoLC and orbitrap mass analyzer using a C18 column and DDA/MS2 mass spectrometer method using the following protocol. Samples were injected (5 μL) onto a reverse phase nanobore HPLC column (AcuTech Scientific, C18, 1.8 μm particle size, 360 μm×20 cm, 150 um ID), equilibrated in solvent E and eluted (500 nL/min) with an increasing concentration of solvent F (acetonitrile/water/FA, 98/2/0.1, v/v/v: min/% F; 0/0, 5/3, 18/7, 74/12, 144/24, 153/27, 162/40, 164/80, 174/80, 176/0, 180/0) using an Easy-nLC II system (Thermo Fisher Scientific). The effluent from the column was directed to a nanospray ionization source connected to a hybrid quadrupole-Orbitrap mass spectrometer (Q Exactive Plus, Thermo Fisher Scientific) operating in the positive ion data-dependent mode alternating between a full scan (m/z 350-1700, automated gain control (AGC) target 3×106, 50 ms maximum injection time, FWHM resolution 70,000 at m/z 200) and up to 10 MS/MS scans (quadrupole isolation of charge states ≥ 2, isolation width 0.7 Th) with previously optimized fragmentation conditions (normalized collision energy of 32, dynamic exclusion of 30 s, AGC target 1×105, 100 ms maximum injection time, FWHM resolution 35,000 at m/z 200). The raw data from both analyses was processed using Proteome Discoverer™ (version 2.4). Tryptic peptides containing amino acid sequences unique to individual proteins were used for identification/quantification of each protein.

In this mass spectrometry experiment, only one phosphosite S50-p was detected to be phosphorylated in beta casein in both 1066 milk and raw milk (data not shown). It is worth noting that phosphorylation was detected in 1066 milk without phosphopeptide enrichment. In raw milk, higher abundance of phosphorylation is detected in alphαS1-casein compared to beta-casein (data not shown). Phosphorylation of alphaSI casein was not detected in 1066 milk (data not shown).

### Example 10. Phosphorylated Caseins Form In Vivo Micelles Within Soy Protein Storage Vacuoles

In this experiment, recombinant versions of alphaSI casein, beta casein, and kappa casein form casein micelles within soy protein storage vacuoles when phosphorylated by BtFam20C kinase. In this example, late stage zygotic soy embryos (14 mm yellow beans/˜50 days post flowering) were transiently transformed via agrobacterium transformation with either pMOZ1066—a plasmid that encodes recombinant versions of Alpha SI casein, Beta casein, Kappa casein, and BtFAM20C kinase that are each respectively tagged with a FLAG epitope and an ER retention signal—or pMOZ1085—the same plasmid as pMOZ1066 but without the BtFAM20C kinase. Although more difficult to transform than younger embryos, late stage embryos were used in order to ensure the formation and deposition of recombinant proteins into soy protein storage vacuoles (PSVs)—a cellular compartment that functions as a protein storage reserve during seed maturation. Once transformed, embryos containing either pMOZ1066 or pMOZ1085 were incubated in a 24° C. growth chamber for 6 days to allow for the optimal expression and visualization of recombinant caseins using transmission electron microscopy.

Tissues from successfully transformed embryos was harvested and fixed using a 0.1% glutaraldehyde solution. Then, each sample was dehydrated using an ethanol series (10%, 30%, 50%, 70%, 90%, 100% and 100%), and embedded in LR White resin. LR white resin was preferred over other resins because it is more amenable to immunolabeling. Once resin blocks solidified, pMOZ1066 and pMOZ1085 samples were ultra-thin sectioned, mounted on carbon-coated TEM grids and immunolabeled with FLAG primary antibodies (1:10) and conjugated Au secondary antibodies (1:30).

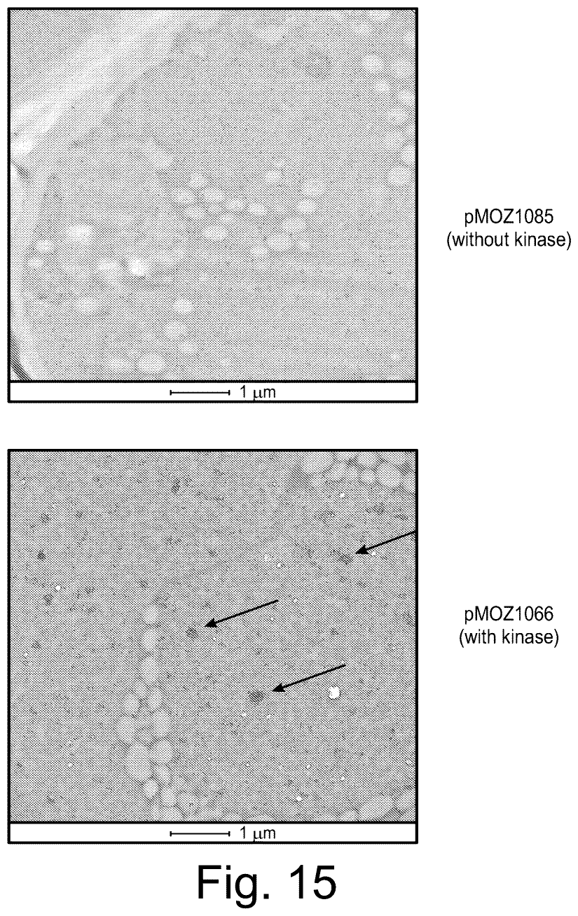

Following immunolabeling, pMOZ1066 and pMOZ1085 samples were imaged using transmission electron microscopy (TEM). In pMOZ1066 samples, casein micelles form within developing PSVs when all three caseins are in the presence of the BtFAM20C kinase (pMOZ1066), shown in FIG. 15 (with arrows pointing towards the casein micelles). However, in pMOZ1085 samples, casein micelles do not form within the PSVs when the BtFAM20C kinase is not present (pMOZ1085), as shown in FIG. 15.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

Claims what is claimed is:

1. A plant cell co-expressing a kinase, a κ-casein, and at least one of a αS1-casein, a αS2-casein, or a β-casein; wherein the κ-casein or at least one of a αS1-casein, a αS2-casein, or a β-casein is phosphorylated by the kinase in the plant cell, and wherein the κ-casein and at least one of a αS1-casein, a αS2-casein, or a β-casein form a casein micelle in the plant cell.

2. A plant cell co-expressing a recombinant kinase, a β-casein, a κ-casein, and a αS1-casein; wherein the β-casein is phosphorylated by the kinase protein in the plant cell; and wherein the phosphorylated β-casein, the κ-casein, and the αS1-casein form a casein micelle in the plant cell.

3. A method of phosphorylating a casein protein, comprising using one or more vectors to co-express a recombinant kinase, a κ-casein, and at least one of a αS1-casein, a αS2-casein, or a β-casein, in a plant cell; wherein at least one of the caseins is phosphorylated by the kinase in the plant cell; and isolating at least one of the phosphorylated caseins from the plant cell.

4. The method in clam 3, wherein the kinase, the κ-casein, and at least one of the αS1-casein, the αS2-casein, or the β-casein are expressed in the plant cell using the same vector.

5. The method in clam 3, wherein the kinase, the κ-casein, and at least one of the αS1-casein, the αS2-casein, or the-casein are expressed in the plant cell using different vectors.

6. The method in any one of claims 3-5, further comprising purifying or enriching the at least one of the phosphorylated caseins isolated from the plant cell.

7. The method in claim 6, wherein the phosphorylated casein enriched is β-casein.

8. The method in any one of claims 3-7, further comprising removing a native plant protein from the isolated proteins.

9. The method of any one of claims 3-8, wherein the kinase has at least 80% sequence identity to Casein Kinase II, a tyrosine kinase, FAM20A, or FAM20C.

10. A composition, comprising:
a deoxyribonucleic acid (DNA) sequence encoding at least a portion of a recombinant kinase protein; and
a recombinant κ-casein and at least one of a recombinant αS1-casein, a recombinant αS2-casein, or a recombinant β-casein.

11. The composition in claim 10, wherein at least one of the recombinant caseins is phosphorylated.

12. The composition in claim 10, wherein the recombinant κ-casein is phosphorylated.

13. The composition in claim 10, wherein the recombinant β-casein is phosphorylated.

14. The composition in claim 13, wherein the recombinant β-casein is phosphorylated at S50.

15. The composition in any one of claims 11-14, wherein the recombinant αS1-casein or the recombinant αS2-casein is phosphorylated.

16. The composition in any one of claims 10-15, wherein the at least one of the recombinant caseins is phosphorylated by a recombinant kinase protein in a plant cell.

17. The composition in claim 16, wherein the composition further comprises the recombinant kinase protein.

18. The composition in any of claims 10-17, wherein the recombinant κ-casein and at least one of a recombinant αS1-casein, a recombinant αS2-casein, or a recombinant β-casein form a casein micelle.

19. The composition in any one of claims 10-18, wherein the deoxyribonucleic acid (DNA) sequence encoding at least the portion of the recombinant kinase protein is at least 50%, at least 60%, at least 70%, at least 80%, at least 90%, at least 95%, or at least 99% of the deoxyribonucleic acid (DNA) sequence encoding the recombinant kinase protein.

20. The composition in any one of claims 10-18, further comprising one or more soy proteins.

21. The composition in claim 20, wherein the one or more soy proteins comprise at least 50%, at least 60%, at least 70%, at least 80%, at least 90%, or at least 95% total protein content by weight in the composition.

22. The composition in claim 20, wherein the one or more soy proteins comprise at most 40%, at most 30%, at most 20%, at most 10%, at most 5%, or at most 1% total protein content by weight in the composition.

23. The composition in claim 20, wherein the one or more soy proteins comprise at most 0.5%, at most 0.4%, at most 0.3%, at most 0.2%, or at most 0.1% of total protein content by weight in the composition.

24. The composition in any one of claims 10-23, wherein the recombinant casein proteins comprise less than 40%, less than 30%, less than 20%, less than 15%, less than 10%, less than 5%, or less than 1% of total protein content by weight in the composition.

25. The composition in any one of claims 10-24, wherein the recombinant-casein is at least 10%, at least 20%, at least 30%, at least 40%, or at least 45% of total casein abundance by weight.

26. The composition in any one of claims 10-24, wherein the recombinant κ-casein is at most 40%, at most 30%, at most 20%, or at most 10% of total casein abundance by weight.

27. The composition in any one of claims 10-24, wherein the recombinant κ-casein is about 46% of total casein abundance by weight.

28. The composition in any one of claims 10-24, wherein the recombinant αS1-casein is less than 60%, or less than 50%, or less than 40% of total casein abundance by weight.

29. The composition in any one of claims 10-24, wherein the recombinant αS1-casein is about 33% of total casein abundance by weight.

30. The composition in any one of claims 10-29, wherein the phosphorylation of recombinant αS1-casein protein is undetectable using mass spectrometry.

31. The composition in any one of claims 10-30, wherein the recombinant kinase protein comprises at least 50%, at least 60%, at least 70%, at least 80%, at least 90%, at least 95%, or at least 99% sequence identity to Casein Kinase II, a tyrosine kinase, FAM20A, or FAM20C.

32. The composition in any one of claims 10-31, further comprises a green fluorescent protein (GFP).

33. The composition in claim 32, wherein the green fluorescent protein (GFP) is less than 1% of total protein content by weight in the composition.

34. The composition in any one of claims 10-33, wherein the deoxyribonucleic acid (DNA) encoding at least the portion of the recombinant kinase protein is present in the composition in trace amounts.

35. The composition in claim 34, wherein the deoxyribonucleic acid (DNA) encoding at least the portion of recombinant kinase protein is detectable using a polymerase chain reaction (PCR) test.

36. The composition in any one of claims 10-35, wherein the composition is essentially free of αS2-casein.

37. A dairy product or dairy product substitute, comprising the composition in any one of claims 10-36.

38. The dairy product or dairy product substitute in claim 37, wherein the dairy product or dairy product substitute resembles raw milk in terms of at least one of physical appearance, sensory perceptions, viscosity, foaming behavior, agglutination capacity, or any combination thereof.

39. The dairy product or dairy product substitute in claim 37 or 38, wherein the dairy product or dairy product substitute is essentially free of milk proteins other than the recombinant casein proteins.

40. A nucleic acid molecule encoding a recombinant kinase protein, a recombinant κ-casein and at least one of a recombinant αS1-casein, a recombinant αS2-casein, or a recombinant β-casein.

41. The nucleic acid molecule of claim 40, wherein the nucleic acid sequence is codon optimized for expression in a plant.

42. The nucleic acid molecule of claim 41, wherein the plant is at least one of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, cactus, turf grass, corn, rice, oat, wheat, barley, sorghum, orchid, iris, lily, onion, palm, or duckweed.

43. The nucleic acid molecule of any one of claims 40-42, wherein the nucleic acid molecule is stably integrated into the genome of a plant cell.

44. An expression vector comprising the nucleic acid molecule of claim 40.

45. A plant tissue comprising the nucleic acid molecule in any one of claims 40-44.

46. The plant tissue in claim 45, wherein the plant tissue is at least one of leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, or leaf sheath of the plant.

47. A nucleic acid molecule for expressing a phosphorylated payload protein in a plant, comprising,
a first polynucleotide sequence encoding a first kinase,
a second polynucleotide sequence encoding a second kinase, and
a third polynucleotide sequence encoding the payload protein.

48. The nucleic acid molecule of claim 47, further comprising a promoter sequence.

49. The nucleic acid molecule of claim 48, wherein the promoter sequence is selected from the group consisting of CaMV 35S, AtuMas Pro+5′UTR, Arabidopsis RPS5a, and RbcS2 promoter.

50. The nucleic acid molecule of claim 47, further comprising a terminator sequence.

51. The nucleic acid molecule of claim 50, wherein the terminator sequence is selected from the group consisting of octopine synthase terminator (Ocst), NOS terminator, and octopine (OCS) terminator.

52. The nucleic acid molecule of claim 47, wherein the third polynucleotide sequence is between the first and second polynucleotide sequences.

53. The nucleic acid molecule of claim 47, wherein the second polynucleotide sequence is between the first and third polynucleotide sequences.

54. The nucleic acid molecule of claim 47, wherein the payload protein is a mammalian protein.

55. The nucleic acid molecule of claim 47, wherein the payload protein is a bovine protein.

56. The nucleic acid molecule of claim 47, wherein the payload protein comprises a whey protein.

57. The nucleic acid molecule of claim 56, wherein the whey protein is selected from the group consisting of α-lactalbumin, β-lactoglobulin, serum albumin, immunoglobulins, and proteose peptone.

58. The nucleic acid molecule of claim 47, wherein the payload protein comprises a collagen protein.

59. The nucleic acid molecule of claim 47, wherein the payload protein comprises an egg white protein.

60. The nucleic acid molecule of claim 59, wherein the egg white protein is selected from the group consisting of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, avidin, and cystatin.

61. The nucleic acid molecule of claim 47, wherein the payload protein comprises a collagen protein comprising at least one of Collagen I, Collagen II, Collagen III, Collagen IV, Collagen V, Collagen VI, Collagen VII, Collagen VIII, Collagen IX, Collagen X, Collagen XI, Collagen XII, Collagen XIII, Collagen XIV, Collagen XV, Collagen XVI, Collagen XVII, Collagen XVIII, Collagen XIX, Collagen XX, Collagen XXI, Collagen XXII, Collagen XXIII, Collagen XXIV, Collagen XXV, Collagen XXVI, Collagen XXVII, and Collagen XXVIII.

62. The nucleic acid molecule of claim 59, wherein the egg white protein comprises at least 85% sequence identity to the amino acid SEQ ID: 10, SEQ ID: 11, SEQ ID: 12, SEQ ID: 13, SEQ ID: 16, SEQ ID: 17, SEQ ID: 18, SEQ ID: 20, SEQ ID: 21, or SEQ ID: 22.

63. The nucleic acid molecule of claim 47, wherein the payload protein comprises a casein protein.

64. The nucleic acid molecule of claim 63, wherein the casein protein comprises at least one of αS1-casein, αS2-casein, β-casein, and κ-casein.

65. The nucleic acid molecule of claim 47, wherein the payload protein comprises at least 85% sequence identity to SEQ ID: 1, SEQ ID: 2, SEQ ID: 3, or SEQ ID: 4.

66. The nucleic acid molecule of any one of claims 47 to 65, further comprising a fourth polynucleotide sequence encoding a second payload protein different from the payload protein, and/or a third payload protein different from the first and second payload protein.

67. The nucleic acid molecule of claim 66, wherein the payload protein comprises κ-casein, and the second and third payload protein comprises at least one of αS1-casein, αS2-casein, or β-casein.

68. The nucleic acid molecule of claim 66, wherein the payload protein comprises at least 85% sequence identity to SEQ ID: 1, and the second payload protein has at least 85% sequence identity to SEQ ID: 2, SEQ ID: 3, or SEQ ID: 4.

69. The nucleic acid molecule of any one of claims 47 to 68, wherein at least one of the first and the second kinase is a human kinase.

70. The nucleic acid molecule of any one of claims 47 to 68, wherein at least one of the first and the second kinase is a bovine kinase.

71. The nucleic acid molecule of any one of claims 47 to 68, wherein at least one of the first and the second kinase has at least 50% sequence identity to SEQ ID: 5 or SEQ ID: 6.

72. The nucleic acid molecule of any one of claims 47 to 68, wherein at least one of the first and the second kinase has at least 60% sequence identity to SEQ ID: 5 or SEQ ID: 6.

73. The nucleic acid molecule of any one of claims 47 to 68, wherein at least one of the first and the second kinase has at least 70% sequence identity to SEQ ID: 5 or SEQ ID: 6.

74. The nucleic acid molecule of any one of claims 47 to 68, wherein at least one of the first and the second kinase has at least 80% sequence identity to SEQ ID: 5 or SEQ ID: 6.

75. The nucleic acid molecule of any one of claims 47 to 68, wherein at least one of the first and the second kinase has at least 90% sequence identity to SEQ ID: 5 or SEQ ID: 6.

76. The nucleic acid molecule of any one of claims 47 to 68, wherein at least one of the first and the second kinase has at least 95% sequence identity to SEQ ID: 5 or SEQ ID: 6.

77. The nucleic acid molecule of any one of claims 47 to 68, wherein at least one of the first and the second kinase has at least 99% sequence identity to SEQ ID: 5 or SEQ ID: 6.

78. The nucleic acid molecule of any one of claims 47 to 68, wherein at least one of the first and the second kinase comprises Casein Kinase II.

79. The nucleic acid molecule of any one of claims 47 to 68, wherein at least one of the first and the second kinase comprises a tyrosine kinase.

80. The nucleic acid molecule of any one of claims 47 to 68, wherein at least one of the first and the second kinase comprises FAM20A.

81. The nucleic acid molecule of any one of claims 47 to 68, wherein at least one of the first and the second kinase comprises FAM20C.

82. The nucleic acid molecule of any one of claims 47 to 68, wherein the first kinase is different from the second kinase.

83. A nucleic acid molecule for expressing a phosphorylated casein protein in a plant, comprising,
a first polynucleotide sequence encoding a first kinase;
a second polynucleotide sequence encoding κ-casein; and
a third polynucleotide sequence encoding at least one of αS1-casein, αS2-casein, and β-casein.

84. The nucleic acid molecule of any one of claims 83, wherein the κ-casein has at least 85% sequence identity to SEQ ID: 1.

85. The nucleic acid molecule of claim 83, wherein the κ-casein is a human protein.

86. The nucleic acid molecule of claim 83, wherein the third polynucleotide sequence encodes human β-casein.

87. The nucleic acid molecule of claim 83, wherein the third polynucleotide sequence encodes αS1-casein having at least 85% sequence identity to SEQ ID: 2.

88. The nucleic acid molecule of claim 83, wherein the third polynucleotide sequence encodes αS2-casein having at least 85% sequence identity to SEQ ID: 3.

89. The nucleic acid molecule of claim 83, wherein the third polynucleotide sequence encodes β-casein having at least 85% sequence identity to SEQ ID: 4.

90. The nucleic acid molecule of any one of claims 83-89, wherein the first kinase has at least 50% sequence identity to SEQ ID: 5 or SEQ ID: 6.

91. The nucleic acid molecule of any one of claims 83-89, wherein the first kinase has at least 60% sequence identity to SEQ ID: 5 or SEQ ID: 6.

92. The nucleic acid molecule of any one of claims 83-89, wherein the first kinase has at least 70% sequence identity to SEQ ID: 5 or SEQ ID: 6.

93. The nucleic acid molecule of any one of claims 83-89, wherein the first kinase has at least 80% sequence identity to SEQ ID: 5 or SEQ ID: 6.

94. The nucleic acid molecule of any one of claims 83-89, wherein the first kinase has at least 90% sequence identity to SEQ ID: 5 or SEQ ID: 6.

95. The nucleic acid molecule of any one of claims 83-89, wherein the first kinase has at least 95% sequence identity to SEQ ID: 5 or SEQ ID: 6.

96. The nucleic acid molecule of any one of claims 83-89, wherein the first kinase has at least 99% sequence identity to SEQ ID: 5 or SEQ ID: 6.

97. The nucleic acid molecule of any one of claims 83-89, wherein the first kinase comprises human Casein Kinase II.

98. The nucleic acid molecule of any one of claims 83-89, wherein the first kinase has at least 80% sequence identity to SEQ ID: 8.

99. The nucleic acid molecule of any one of claims 83-89, wherein the first kinase comprises a tyrosine kinase.

100. The nucleic acid molecule of any one of claims 83-89, wherein the first kinase comprises FAM20A.

101. The nucleic acid molecule of any one of claims 83-89, wherein the first kinase comprises FAM20C.

102. The nucleic acid molecule in any one of claims 47 to 101, wherein nucleic acid molecule is stably integrated into the genome of a plant cell.

103. An expression vector comprising the nucleic acid molecule any one of claims 47 to 102.

104. A plant transformed with the expression vector in claim 103, wherein the payload protein is phosphorylated by the first or the second kinase in vivo.

105. The plant in claim 104, wherein the plant is a dicot plant selected from the group consisting of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus.

106. The plant in claim 104, wherein the plant is a monocot plant selected from the group consisting of turf grass, corn, rice, oat, wheat, barley, sorghum, orchid, iris, lily, onion, palm, and duckweed.

107. A plant tissue comprising the nucleic acid molecule in any one of claims 47 to 102, where the plant tissue is at least one of leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, or leaf sheath of the plant.

108. A method for expressing a phosphorylated payload protein in a plant, comprising:
transforming the plant with the nucleic acid molecule in any one of claims 47 to 102, and growing the transformed plant, wherein the payload protein is phosphorylated by the first or second kinase.

109. A method of expressing a phosphorylated payload protein in a plant, comprising:
transforming the plant with a first vector, a second vector, and a third vector; and
growing the transformed plant, wherein the payload protein is phosphorylated by the kinase;
wherein the first vector comprises a first polynucleotide sequence encoding a first kinase,
the second vector comprises a second polynucleotide sequence encoding a second kinase, and
the third vector comprises a third polynucleotide sequence encoding the payload protein.

110. The method of claim 109, wherein the payload protein is a mammal protein.

111. The method of claim 109, wherein the payload protein is a bovine protein.

112. The method of claim 109, wherein the payload protein comprises a whey protein.

113. The method of claim 112, wherein the whey protein is selected from the group consisting of α-lactalbumin, β-lactoglobulin, serum albumin, immunoglobulins, and proteose peptone.

114. The method of claim 109, wherein the payload protein comprises a collagen protein comprising at least one of Collagen I, Collagen II, Collagen III, Collagen IV, Collagen V, Collagen VI, Collagen VII, Collagen VIII, Collagen IX, Collagen X, Collagen XI, Collagen XII, Collagen XIII, Collagen XIV, Collagen XV, Collagen XVI, Collagen XVII, Collagen XVIII, Collagen XIX, Collagen XX, Collagen XXI, Collagen XXII, Collagen XXIII, Collagen XXIV, Collagen XXV, Collagen XXVI, Collagen XXVII, and Collagen XXVIII.

115. The method of claim 109, wherein the payload protein comprises an egg white protein.

116. The method of claim 115, wherein the egg white protein is selected from the group consisting of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, avidin, and cystatin.

117. The method of claim 115, wherein the egg white protein has at least 85% sequence identity to the amino acid SEQ ID: 10, SEQ ID: 11, SEQ ID: 12, SEQ ID: 13, SEQ ID: 16, SEQ ID: 17, SEQ ID: 18, SEQ ID: 20, SEQ ID: 21, or SEQ ID: 22.

118. The method of claim 109, wherein the payload protein comprises a casein protein.

119. The method of claim 118, wherein the casein protein comprises at least one of αS1-casein, αS2-casein, β-casein, and κ-casein.

120. The method of claim 109, wherein the payload protein has at least 85% sequence identity to SEQ ID: 1, SEQ ID: 2, SEQ ID: 3 or SEQ ID: 4.

121. The method in claim 109, further comprising transforming the plant with a fourth vector, wherein the fourth vector comprises a fourth polynucleotide sequence encoding a second payload protein.

122. The method of claim 121, wherein the payload protein has at least 85% sequence identity to SEQ ID: 1, and the second payload protein has at least 85% sequence identity to SEQ ID: 2, SEQ ID: 3, or SEQ ID: 4.

123. The method in claim 121, wherein the payload protein comprises κ-casein, and the second payload protein comprises at least one of αS1-casein, αS2-casein, and β-casein.

124. A method of enhancing casein micelle formation in a plant, comprising:
transforming the plant with a vector in any one of claims 64, 67, and 83 to 89; and
growing the transformed plant;
wherein at least one of κ-casein, αS1-casein, αS2-casein, and β-casein is phosphorylated by the kinase; and
wherein the κ-casein and at least one of αS1-casein, αS2-casein, and β-casein form the casein micelle in the plant in vivo.

125. A method of enhancing a casein micelle formation in a plant, comprising:
transforming the plant with a first vector and a second vector; and
growing the transformed plant;
wherein the first vector comprises a first polynucleotide sequence encoding a kinase, wherein the second vector comprises a second polynucleotide sequence encoding a κ-casein and at least one of αS1-casein, αS2-casein, and β-casein;
wherein at least one of κ-casein, αS1-casein, αS2-casein, and β-casein is phosphorylated by the kinase, and
wherein the κ-casein and at least one of αS1-casein, αS2-casein, and β-casein form the casein micelle in the plant in vivo.

126. A method of enhancing a casein micelle formation in a plant, comprising:
transforming the plant with a first vector, a second vector, and a third vector; and
growing the transformed plant;
wherein the first vector comprises a first polynucleotide sequence encoding a kinase,
wherein the second vector comprises a second polynucleotide sequence encoding a κ-casein,
wherein the third vector comprises a third polynucleotide sequence encoding at least one of αS1-casein, αS2-casein, and β-casein;
wherein at least one of κ-casein, αS1-casein, αS2-casein, and β-casein is phosphorylated by the kinase, and
wherein the κ-casein and at least one of αS1-casein, αS2-casein, and β-casein form the casein micelle in the plant in vivo.

127. The method of any one of claims 106-126, wherein at least one of the first and the second kinase is a human kinase.

128. The method of any one of claims 106-126, wherein at least one of the first and the second kinase is a bovine kinase.

129. The method of any one of claims 106-126, wherein at least one of the first and the second kinase has at least 50% sequence identity to SEQ ID: 5 or SEQ ID: 6.

130. The method of any one of claims 106-126, wherein at least one of the first and the second kinase has at least 60% sequence identity to SEQ ID: 5 or SEQ ID: 6.

131. The method of any one of claims 106-126, wherein at least one of the first and the second kinase has at least 70% sequence identity to SEQ ID: 5 or SEQ ID: 6.

132. The method of any one of claims 106-126, wherein at least one of the first and the second kinase has at least 80% sequence identity to SEQ ID: 5 or SEQ ID: 6.

133. The method of any one of claims 106-126, wherein at least one of the first and the second kinase has at least 90% sequence identity to SEQ ID: 5 or SEQ ID: 6.

134. The method of any one of claims 106-126, wherein at least one of the first and the second kinase has at least 95% sequence identity to SEQ ID: 5 or SEQ ID: 6.

135. The method of any one of claims 106-126, wherein at least one of the first and the second kinase has at least 99% sequence identity to SEQ ID: 5 or SEQ ID: 6.

136. The method of any one of claims 106-126, wherein at least one of the first and the second kinase comprises Casein Kinase II.

137. The method of any one of claims 106-126, wherein at least one of the first and the second kinase comprises a tyrosine kinase.

138. The method of any one of claims 106-126, wherein at least one of the first and the second kinase comprises FAM20A.

139. The method of any one of claims 106-126, wherein at least one of the first and the second kinase comprises FAM20 C.

140. The method of any one of claims 106-126, wherein the first kinase is different from the second kinase.

141. The method of any one of claims 124-140, wherein phosphorylation of a casein protein increases the expression level of the casein protein in the plant, wherein the casein protein is selected form the group consisting of κ-casein, αS1-casein, αS2-casein, and β-casein.

142. The method of claim 141, wherein phosphorylation of a casein protein increases expression level of the casein protein by at least 10%.

143. The method of claim 141, wherein phosphorylation of a casein protein increases expression level of the casein protein by at least 20%.

144. The method of any one of claims 124-140, wherein phosphorylation of a casein protein increases its ability to aggregate or bind to another casein protein, wherein the casein protein is selected form the group consisting of κ-casein, αS1-casein, αS2-casein, and β-casein.

145. The method of any one of claims 124-140, wherein phosphorylation of a casein protein improves casein micelle formation.

146. The method of any one of claims 124-140, wherein phosphorylation of a casein protein increases its binding to calcium.

147. A food product or food product substitute, comprising a phosphorylated payload protein produced using the composition, nucleic acid, method, or plant in any one of the proceeding claims 1-146.

148. The food product or food product substitute in claim 147, wherein the food product or food product substitute is a liquid, wherein phosphorylation of a payload protein or a casein protein increases viscosity of the liquid, and wherein the casein protein is selected form the group consisting of κ-casein, αS1-casein, αS2-casein, and β-casein.

149. The food product or food product substitute in claim 147, wherein the food product or food product substitute comprises at least one of milk, cheese, ice cream, yogurt, cream, butter, protein powder, protein bar, and baby formula.

150. The food product or food product substitute in claim 147, wherein the food product or food product substitute comprises a dairy product or a product that resembles a dairy product.

151. The dairy product or a product that resembles a dairy product in claim 150, wherein the food product or food product substitute comprises at least one of milk, partly skimmed milk, skim milk, cooking milk, condensed milk, flavored milk, goat milk, sheep milk, dried milk, evaporated milk, and milk foam.

152. The food product or food product substitute in claim 147, wherein the food product or food product substitute comprises a product derived from milk, comprising at least one of yogurt, low-fat yogurt, nonfat yogurt, greek yogurt, whipped yogurt, goat milk yogurt, Labneh (labne), sheep milk yogurt, yogurt drink, Lassi, cheese, dairy-based sauce, dairy spread, cream, frozen confections, dairy desserts, butter, dairy powders, infant formula, milk protein concentrate, milk protein isolate, milk protein concentrate, whey protein isolate, demineralized whey protein concentrate, demineralized whey protein concentrate, beta-lactoglobulin concentrate, beta-lactoglobulin isolate, alpha-lactalbumin concentrate, alpha-lactalbumin isolate, glycomacropeptide concentrate, glycomacropeptide isolate, casein concentrate, casein isolate, nutritional supplements, ready-to-drink or ready-to-mix product, pudding, gel, chewable, crisp, and bar.
